(19) United States
(12) Patent Application Publication
(10) Pub. No.: US 2025/0051735 A1
(43) Pub. Date: Feb. 13, 2025

(54) NEXT GENERATION TRANSPOSOSOMES

(71) Applicants: VANDERBILT UNIVERSITY, Nashville, TN (US); The United States of America, as Represented by The Secretary, Department of Health and Human Servic, Bethesda, MD (US)

(72) Inventors: Matthew H. Wilson, Nashville, TN (US); Frederick Dyda, Bethesda, VA (US); Alison B. Hickman, Bethesda, MD (US); Wentian Lou, Nashville, TN (US)

(21) Appl. No.: 18/720,218

(22) PCT Filed: Dec. 22, 2022
(86) PCT No.: PCT/US2022/082217

§ 371 Date: Jun. 14, 2024

Related U.S. Application Data
(60) Provisional application No. 63/292,821, filed on Dec. 22, 2021.

(51) Int. Cl. C12N 9/12; C12N 15/62

(52) U.S. Cl. CPC C12N 9/1241; C12N 15/62; C07K 2319/00; C12Y 207/07

(57) ABSTRACT

Disclosed herein is a modified transposase, comprising a core piggy Bac transposase having one or more modifications making it capable of excising and integrating a piggy Bac transposon in LE/LE configuration leading to hyperactivity. Also disclosed is a modified piggy Bac transposase capable of excising but not integrating a piggy Bac transposon in LE/LE configuration. Also disclosed herein is a modification of the mammalian Myotis lucifugus DNA transposon involving modification both its transposase and transposon LE and RE ends by truncations leading to hyperactivity.

### CROSS-REFERENCE TO RELATED APPLICATIONS

This application is a U.S. National Stage patent application claiming priority to PCT/US2022/082217 filed Dec. 22, 2022, which claims benefit of U.S. Provisional Application No. 63/292,821, filed Dec. 22, 2021, which is are hereby incorporated herein by reference in their its entirety.

### STATEMENT OF GOVERNMENT INTEREST

This invention was made with Government Support under Grant No. DK093660 awarded by the National Institutes of Health. The Government has certain rights in the invention.

### SEQUENCE LISTING

This application contains a sequence listing filed in ST.26 format entitled “222230-1350 Sequence Listing” created on Jul. 19, 2024, having 101,460 bytes. The content of the sequence listing is incorporated herein in its entirety.

### BACKGROUND OF THE INVENTION

Typical methods for introducing DNA into a cell include DNA condensing reagents such as calcium phosphate, polyethylene glycol, lipid-containing reagents, such as liposomes, multi-lamellar vesicles, as well as virus-mediated strategies. However, such methods can have certain limitations. For example, there are size constraints associated with DNA condensing reagents and virus-mediated strategies. Further, the amount of nucleic acid that can be transfected into a cell is limited in virus strategies. Not all methods facilitate insertion of the delivered nucleic acid into cellular nucleic acid, and while DNA condensing methods and lipid-containing reagents are relatively easy to prepare, the insertion of nucleic acid into viral vectors can be labor intensive. Virus-mediated strategies can be cell-type or tissue-type specific, and the use of virus-mediated strategies can create immunologic problems when used in vivo.

One suitable tool to address these issues are transposons. Transposons, or transposable elements, include a (short) nucleic acid sequence, with terminal repeat sequences upstream and downstream. Active transposons encode enzymes that facilitate the excision and insertion of the nucleic acid into target DNA sequences. Both invertebrate and vertebrate transposons hold potential for transgenesis and insertional mutagenesis in model organisms. Particularly, the availability of alternative transposon systems in the same species opens up new possibilities for genetic analyses.

There still remains a need for new methods for introducing DNA into a cell, and particularly methods that promote the efficient insertion of transposons of varying sizes into the nucleic acid of a cell or the insertion of DNA into the genome of a cell while allowing more efficient transcription/translation results than constructs as available in the state of the art.

### SUMMARY OF THE INVENTION

Disclosed herein is a rationally engineered hyperactive piggyBac transposase that can transposition a transposon with LE/LE configuration (shortened inverted repeats (IR) from left end (LE) sequences on both ends). Without wishing to be bound by theory, these engineered transposases may be hyperactive due to a simpler interaction with transposon ends enabling LE/LE interaction and leading to hyperactivity. This new transposase has wide utility for genome engineering, gene transfer, and gene therapy applications.

In some embodiments, the disclosed system can be used to make stable cell lines, transgenic animals, recombinant proteins, engineering iPS cells, cell therapy, CAR-T cell engineering, gene therapy, genome engineering, hybrid transposase-viral vectors, or any combination thereof. Currently, both piggy Bac and sleeping beauty are used for CAR-T cell generation in clinical trials. This new transposase would be more efficient than both of those and could be additionally engineered for targeted integration. This would be a significant advance for the field.

Disclosed herein is a modified transposase, comprising a core piggyBac transposase having one or more modifications making it capable of excising a piggyBac transposon in LE/LE configuration.

In some embodiments, the core piggy Bac transposase is the piggy Bac transposase from Trichoplusia ni (cabbage looper moth). For example, in some embodiments, core piggy Bac transposase has the amino acid sequence: MGSSLDDEHILSALLQSDDELVGEDSDSEISDHVSEDDVQSDTEEAFIDEVHEVQPTSSGSEIL DEQNVIEQPGSSLASNRILTLPQRTIRGKNKHCWSTSKSTRRSRVSALNIVRSQRGPTRMCRNI YDPLLCFKLFFTDEIISEIVKWTNAEISLKRRESMTGATFRDTNEDEIYAFFGILVMTAVRKDNHM STDDLFDRSLSMVYVSVMSRDRFDFLIRCLRMDDKSIRPTLRENDVFTPVRKIWDLFIHQCIQNY TPGAHLTIDEQLLGFRGRCPFRMYIPNKPSKYGIKILMMCDSGTKYMINGMPYLGRGTQTNGVP LGEYYVKELSKPVHGSCRNITCDNWFTSIPLAKNLLQEPYKLTIVGTVRSNKREIPEVLKNSRSR PVGTSMFCFDGPLTLVSYKPKPAKMVYLLSSCDEDASINESTGKPQMVMYYNQTKGGVDTLD QMCSVMTCSRKTNRWPMALLYGMINIACINSFIIYSHNVSSKGEKVQSRKKFMRNLYMSLTSSF MRKRLEAPTLKRYLRDNISNILPNEVPGTSDDSTEEPVMKKRTYCTYCPSKIRRKANASCKKCK KVICREHNIDMCQSCF (SEQ ID NO:1). The underlined N-terminal 73 amino acids are shown herein to be unnecessary for LE:LE binding, and the underlined C-terminal 53 amino acids are shown herein to be involved in LE:LE binding.

Therefore, in some embodiments, the disclosed transposase lacks one or more of the N-terminal 74 amino acids. Therefore, in some embodiments, the disclosed piggy Bac transposase has the amino acid sequence: Xₐ-SSLASNRILTLPQRTIRGKNKHCWSTSKSTRRSRVSALNIVRSQRGPTRMCRNIYDPLLCF KLFFTDEIISEIVKWTNAEISLKRRESMTGATFRDTNEDEIYAFFGILVMTAVRKDNHMSTDDLFD RSLSMVYVSVMSRDRFDFLIRCLRMDDKSIRPTLRENDVFTPVRKIWDLFIHQCIQNYTPGAHLT IDEQLLGFRGRCPFRMYIPNKPSKYGIKILMMCDSGTKYMINGMPYLGRGTQTNGVPLGEYYV KELSKPVHGSCRNITCDNWFTSIPLAKNLLQEPYKLTIVGTVRSNKREIPEVLKNSRSRPVGTSM FCFDGPLTLVSYKPKPAKMVYLLSSCDEDASINESTGKPQMVMYYNQTKGGVDTLDQMCSVM TCSRKTNRWPMALLYGMINIACINSFIIYSHNVSSKGEKVQSRKKFMRNLYMSLTSSFMRKRLE APTLKRYLRDNISNILPNEVPGTSDDSTEEPVMKKRTYCTYCPSKIRRKANASCKKCKKVICREH NIDMCQSCF (SEQ ID NO:2), wherein Xₐ is 2 to 74 aa of the amino acid sequence:

(SEQ ID NO: 3)
MGSSLDDEHILSALLQSDDELVGEDSDSEISDHVSEDDVQSDTEEAFID
EVHEVQPTSSGSEILDEQNVIEQPG.

Therefore, in some embodiments, the disclosed piggy Bac transposase has the amino acid sequence: MSSLASNRILTLPQRTIRGKNKHCWSTSKSTRRSRVSALNIVRSQRGPTRMCRNIYDPLLCFKL FFTDEIISEIVKWTNAEISLKRRESMTGATFRDTNEDEIYAFFGILVMTAVRKDNHMSTDDLFDRS LSMVYVSVMSRDRFDFLIRCLRMDDKSIRPTLRENDVFTPVRKIWDLFIHQCIQNYTPGAHLTID EQLLGFRGRCPFRMYIPNKPSKYGIKILMMCDSGTKYMINGMPYLGRGTQTNGVPLGEYYVKE LSKPVHGSCRNITCDNWFTSIPLAKNLLQEPYKLTIVGTVRSNKREIPEVLKNSRSRPVGTSMFC FDGPLTLVSYKPKPAKMVYLLSSCDEDASINESTGKPQMVMYYNQTKGGVDTLDQMCSVMTC SRKTNRWPMALLYGMINIACINSFIIYSHNVSSKGEKVQSRKKFMRNLYMSLTSSFMRKRLEAP TLKRYLRDNISNILPNEVPGTSDDSTEEPVMKKRTYCTYCPSKIRRKANASCKKCKKVICREHNI DMCQSCF (SEQ ID NO:4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 4.

Therefore, in some embodiments, the disclosed transposase has a tandem additional C-terminal domain. Therefore, in some embodiments, the disclosed piggy Bac transposase has the amino acid sequence: MGSSLDDEHILSALLQSDDELVGEDSDSEISDHVSEDDVQSDTEEAFIDEVHEVQPTSSGSEIL DEQNVIEQPGSSLASNRILTLPQRTIRGKNKHCWSTSKSTRRSRVSALNIVRSQRGPTRMCRNI YDPLLCFKLFFTDEIISEIVKWTNAEISLKRRESMTGATFRDTNEDEIYAFFGILVMTAVRKDNHM STDDLFDRSLSMVYVSVMSRDRFDFLIRCLRMDDKSIRPTLRENDVFTPVRKIWDLFIHQCIQNY TPGAHLTIDEQLLGFRGRCPFRMYIPNKPSKYGIKILMMCDSGTKYMINGMPYLGRGTQTNGVP LGEYYVKELSKPVHGSCRNITCDNWFTSIPLAKNLLQEPYKLTIVGTVRSNKREIPEVLKNSRSR PVGTSMFCFDGPLTLVSYKPKPAKMVYLLSSCDEDASINESTGKPQMVMYYNQTKGGVDTLD QMCSVMTCSRKTNRWPMALLYGMINIACINSFIIYSHNVSSKGEKVQSRKKFMRNLYMSLTSSF MRKRLEAPTLKRYLRDNISNILPNEVPGTSDDSTEEPVMKKRTYCTYCPSKIRRKANASCKKCK KVICREHNIDMCQSCF (SEQ ID NO:1)-X₀-X, wherein X_{b} is 40-53 aa of the amino acid sequence GTSDDSTEEPVMKKRTYCTYCPSKIRRKANASCKKCKKVICREHNIDMCQSCF (SEQ ID NO:5), and wherein X₀ is a linker comprising 0-20 amino acid residues.

In some embodiments, the disclosed transposase lacks one or more of the terminal 73 amino acids and has a tandem additional C-terminal domain. Therefore, in some embodiments, the disclosed piggyBac transposase has the amino acid sequence: Xₐ-SSLASNRILTLPQRTIRGKNKHCWSTSKSTRRSRVSALNIVRSQRGPTRMCRNIYDPLLCFKLFF TDEIISEIVKWTNAEISLKRRESMTGATFRDTNEDEIYAFFGILVMTAVRKDNHMSTDDLFDRSLS MVYVSVMSRDRFDFLIRCLRMDDKSIRPTLRENDVFTPVRKIWDLFIHQCIQNYTPGAHLTIDEQ LLGFRGRCPFRMYIPNKPSKYGIKILMMCDSGTKYMINGMPYLGRGTQTNGVPLGEYYVKELS KPVHGSCRNITCDNWFTSIPLAKNLLQEPYKLTIVGTVRSNKREIPEVLKNSRSRPVGTSMFCF DGPLTLVSYKPKPAKMVYLLSSCDEDASINESTGKPQMVMYYNQTKGGVDTLDQMCSVMTCS RKTNRWPMALLYGMINIACINSFIIYSHNVSSKGEKVQSRKKFMRNLYMSLTSSFMRKRLEAPT LKRYLRDNISNILPNEVPGTSDDSTEEPVMKKRTYCTYCPSKIRRKANASCKKCKKVICREHNID MCQSCF (SEQ ID NO:2)-X₀-Xb wherein Xₐ is 2 to 74 aa of the amino acid sequence: MGSSLDDEHILSALLQSDDELVGEDSDSEISDHVSEDDVQSDTEEAFIDEVHEVQPTSSGSEIL DEQNVIEQPG (SEQ ID NO:3), wherein X_{b} is 40-53 aa of the amino acid sequence GTSDDSTEEPVMKKRTYCTYCPSKIRRKANASCKKCKKVICREHNIDMCQSCF (SEQ ID NO:5), and wherein X₀ is a linker comprising 0-20 amino acid residues.

Therefore, in some embodiments, the disclosed piggy Bac transposase has the amino acid sequence: MSSLASNRILTLPQRTIRGKNKHCWSTSKSTRRSRVSALNIVRSQRGPTRMCRNIYDPLLCFKL FFTDEIISEIVKWTNAEISLKRRESMTGATFRDTNEDEIYAFFGILVMTAVRKDNHMSTDDLFDRS LSMVYVSVMSRDRFDFLIRCLRMDDKSIRPTLRENDVFTPVRKIWDLFIHQCIQNYTPGAHLTID EQLLGFRGRCPFRMYIPNKPSKYGIKILMMCDSGTKYMINGMPYLGRGTQTNGVPLGEYYVKE LSKPVHGSCRNITCDNWFTSIPLAKNLLQEPYKLTIVGTVRSNKREIPEVLKNSRSRPVGTSMFC FDGPLTLVSYKPKPAKMVYLLSSCDEDASINESTGKPQMVMYYNQTKGGVDTLDQMCSVMTC SRKTNRWPMALLYGMINIACINSFIIYSHNVSSKGEKVQSRKKFMRNLYMSLTSSFMRKRLEAP TLKRYLRDNISNILPNEVPGTSDDSTEEPVMKKRTYCTYCPSKIRRKANASCKKCKKVICREHNI DMCQSCFGTSDDSTEEPVMKKRTYCTYCPSKIRRKANASCKKCKKVICREHNIDMCQSCF (SEQ ID NO:6), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:8.

In some embodiments, the core piggyBac transposase is the hyperactive transposase, such as those described in U.S. Pat. No. 9,670,503, which is incorporated by reference for the teaching of these transposases and transposons. Examples of hyperactive piggy Bac transposase mutations include: G2C, Q40R, S3N, S26P, I30V, G165S, T43A, Q55R, T57A, S61R, I82V, I90V, S103P, S103T, N113S, M185L, M194V, S230N, R281G, M282V, G316E, P410L, I426V, Q497L, K501N, N505D, X₅₀₉G, S509G, N538K, N538K, N570S, S573L, K565I, K575R, Q591P, Q591R, and F594L.

For example, in some embodiments, core piggyBac transposase has the amino acid sequence: X₁SX₂LDDEHILSALLQSDDELVGEDX₃DSEX₄SDHVSEDDVX₅SDX₆EEAFIDEVHEVX₇PX₈SSG X₉EILDEQNVIEQPGSSLASNRX₁₀LTLPQRTX₁₁RGKNKHCWSTSKX₁₂TRRSRVSALX₁₃IVRSQR GPTRMCRNIYDPLLCFKLFFTDEIISEIVKWTNAEISLKRRESMTX₁₄ATFRDTNEDEIYAFFGILVX₁₅TAVRKDNHX₁₆STDDLFDRSLSMVYVSVMSRDRFDFLIRCLRMDDKX₁₇IRPTLRENDVFTPVR KIWDLFIHQCIQNYTPGAHLTIDEQLLGFRGRCPFX₁₈X₁₉YIPNKPSKYGIKILMMCDSGTKYMIN GMPYLGRX₂₀TQTNGVPLGEYYVKELSKPVHGSCRNITCDNWFTSIPLAKNLLQEPYKLTIVGTV RSNKREIPEVLKNSRSRPVGTSMFCFDGPLTLVSYKPKX₂₁AKMVYLLSSCDEDASX₂₂NESTGK PQMVMYYNQTKGGVDTLDQMCSVMTCSRKTNRWPMALLYGMINIACINSFIIYSHNVSSKGEK VX₂₃SRKX₂₄FMRX₂₅LYMX₂₆LTSSFMRKRLEAPTLKRYLRDNISNILPX₂₇EVPGTSDDSTEEPVMK KRTYCTYCPSX₂₈IRRKAX₂₉AX₃₀CX₃₁KCKKVICREHNIDMCX₃₂SCX₃₃ (SEQ ID NO:7), wherein X₁ is G or C, wherein X₂ is S or N, wherein X₃ is S or P, wherein X₄ is I or V, wherein X₅ is Q or R, wherein X₆ is T or A, wherein X₇ is Q or R, wherein X₈ is T or A, wherein X₉ is S or R, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

In some embodiments, the core piggyBac transposase has the amino acid sequence:

(SEQ ID NO: 8, R372A)
MGSSLDDEHILSALLQSDDELVGEDSDSEISDHVSEDDVQSDTEEAFID
EVHEVQPTSSGSEILDEQNVIEQPGSSLASNRILTLPQRTIRGKNKHCW
STSKSTRRSRVSALNIVRSQRGPTRMCRNIYDPLLCFKLFFTDEIISEI
VKWTNAEISLKRRESMTGATFRDTNEDEIYAFFGILVMTAVRKDNHMST
DDLFDRSLSMVYVSVMSRDRFDFLIRCLRMDDKSIRPTLRENDVFTPVR
KIWDLFIHQCIQNYTPGAHLTIDEQLLGFRGRCPFRMYIPNKPSKYGIK
ILMMCDSGTKYMINGMPYLGRGTQTNGVPLGEYYVKELSKPVHGSCRNI
TCDNWFTSIPLAKNLLQEPYKLTIVGTVASNKREIPEVLKNSRSRPVGT
SMFCFDGPLTLVSYKPKPAKMVYLLSSCDEDASINESTGKPQMVMYYNQ
TKGGVDTLDQMCSVMTCSRKTNRWPMALLYGMINIACINSFIIYSHNVS
SKGEKVQSRKKFMRNLYMSLTSSFMRKRLEAPTLKRYLRDNISNILPNE
VPGTSDDSTEEPVMKKRTYCTYCPSKIRRKANASCKKCKKVICREHNID
MCQSCF.

In some embodiments, the core piggyBac transposase has the amino acid sequence:

(SEQ ID NO: 9, K375A)
MGSSLDDEHILSALLQSDDELVGEDSDSEISDHVSEDDVQSDTEEAFID
EVHEVQPTSSGSEILDEQNVIEQPGSSLASNRILTLPQRTIRGKNKHCW
STSKSTRRSRVSALNIVRSQRGPTRMCRNIYDPLLCFKLFFTDEIISEI
VKWTNAEISLKRRESMTGATFRDTNEDEIYAFFGILVMTAVRKDNHMST
DDLFDRSLSMVYVSVMSRDRFDFLIRCLRMDDKSIRPTLRENDVFTPVR
KIWDLFIHQCIQNYTPGAHLTIDEQLLGFRGRCPFRMYIPNKPSKYGIK
ILMMCDSGTKYMINGMPYLGRGTQTNGVPLGEYYVKELSKPVHGSCRNI
TCDNWFTSIPLAKNLLQEPYKLTIVGTVRSNAREIPEVLKNSRSRPVGT
SMFCFDGPLTLVSYKPKPAKMVYLLSSCDEDASINESTGKPQMVMYYNQ
TKGGVDTLDQMCSVMTCSRKTNRWPMALLYGMINIACINSFIIYSHNVS
SKGEKVQSRKKFMRNLYMSLTSSFMRKRLEAPTLKRYLRDNISNILPNE
VPGTSDDSTEEPVMKKRTYCTYCPSKIRRKANASCKKCKKVICREHNID
MCQSCF.

In some embodiments, the core piggyBac transposase has the amino acid sequence:

(SEQ ID NO: 10, R372A, K375A)
MGSSLDDEHILSALLQSDDELVGEDSDSEISDHVSEDDVQSDTEEAFID
EVHEVQPTSSGSEILDEQNVIEQPGSSLASNRILTLPQRTIRGKNKHCW
STSKSTRRSRVSALNIVRSQRGPTRMCRNIYDPLLCFKLFFTDEIISEI
VKWTNAEISLKRRESMTGATFRDTNEDEIYAFFGILVMTAVRKDNHMST
DDLFDRSLSMVYVSVMSRDRFDFLIRCLRMDDKSIRPTLRENDVFTPVR
KIWDLFIHQCIQNYTPGAHLTIDEQLLGFRGRCPFRMYIPNKPSKYGIK
ILMMCDSGTKYMINGMPYLGRGTQTNGVPLGEYYVKELSKPVHGSCRNI
TCDNWFTSIPLAKNLLQEPYKLTIVGTVASNAREIPEVLKNSRSRPVGT
SMFCFDGPLTLVSYKPKPAKMVYLLSSCDEDASINESTGKPQMVMYYNQ
TKGGVDTLDQMCSVMTCSRKTNRWPMALLYGMINIACINSFIIYSHNVS
SKGEKVQSRKKFMRNLYMSLTSSFMRKRLEAPTLKRYLRDNISNILPNE
VPGTSDDSTEEPVMKKRTYCTYCPSKIRRKANASCKKCKKVICREHNID
MCQSCF.

Therefore, in some embodiments, piggyBac transposase has the amino acid sequence: MSSLASNRX₁₀LTLPQRTX₁₁RGKNKHCWSTSKX₁₂TRRSRVSALX₁₃IVRSQRGPTRMCRNIYDP LLCFKLFFTDEIISEIVKWTNAEISLKRRESMTX₁₄ATFRDTNEDEIYAFFGILVX₁₅TAVRKDNHX₁₆S TDDLFDRSLSMVYVSVMSRDRFDFLIRCLRMDDKX₁₇IRPTLRENDVFTPVRKIWDLFIHQCIQN YTPGAHLTIDEQLLGFRGRCPFX₁₈X₁₉YIPNKPSKYGIKILMMCDSGTKYMINGMPYLGRX₂₀TQT NGVPLGEYYVKELSKPVHGSCRNITCDNWFTSIPLAKNLLQEPYKLTIVGTVRSNKREIPEVLKN SRSRPVGTSMFCFDGPLTLVSYKPKX₂₁AKMVYLLSSCDEDASX₂₂NESTGKPQMVMYYNQTKG GVDTLDQMCSVMTCSRKTNRWPMALLYGMINIACINSFIIYSHNVSSKGEKVX₂₃SRKX₂₄FMRX₂₅LYMX₂₆LTSSFMRKRLEAPTLKRYLRDNISNILPX₂₇EVPGTSDDSTEEPVMKKRTYCTYCPSX₂₈I RRKAX₂₉AX₃₀CX₃₁KCKKVICREHNIDMCX₃₂SCX₃₃ (SEQ ID NO:11), wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

In some embodiments, core piggy Bac transposase has the amino acid sequence: X₁SX₂LDDEHILSALLQSDDELVGEDX₃DSEX₄SDHVSEDDVX₅SDX₆EEAFIDEVHEVX₇PX₈SSG X₉EILDEQNVIEQPGSSLASNRX₁₀LTLPQRTX₁₁RGKNKHCWSTSKX₁₂TRRSRVSALX₁₃IVRSQR GPTRMCRNIYDPLLCFKLFFTDEIISEIVKWTNAEISLKRRESMTX₁₄ATFRDTNEDEIYAFFGILVX₁₅TAVRKDNHX₁₆STDDLFDRSLSMVYVSVMSRDRFDFLIRCLRMDDKX₁₇IRPTLRENDVFTPVR KIWDLFIHQCIQNYTPGAHLTIDEQLLGFRGRCPFX₁₈X₁₉YIPNKPSKYGIKILMMCDSGTKYMIN GMPYLGRX₂₀TQTNGVPLGEYYVKELSKPVHGSCRNITCDNWFTSIPLAKNLLQEPYKLTIVGTV RSNKREIPEVLKNSRSRPVGTSMFCFDGPLTLVSYKPKX₂₁AKMVYLLSSCDEDASX₂₂NESTGK PQMVMYYNQTKGGVDTLDQMCSVMTCSRKTNRWPMALLYGMINIACINSFIIYSHNVSSKGEK VX₂₃SRKX₂₄FMRX₂₅LYMX₂₆LTSSFMRKRLEAPTLKRYLRDNISNILPX₂₇EVPGTSDDSTEEPVMK KRTYCTYCPSX₂₈IRRKAX₂₉AX₃₀CX₃₁KCKKVICREHNIDMCX₃₂SCX₃₃GTSDDSTEEPVMKKRTY CTYCPSX₂₈IRRKAX₂₉AX₃₀CX₃₁KCKKVICREHNIDMCX₃₂SCX₃₃ (SEQ ID NO:12), wherein X₁ is G or C, wherein X₂ is S or N, wherein X₃ is S or P, wherein X₄ is I or V, wherein X₅ is Q or R, wherein X₆ is T or A, wherein X₇ is Q or R, wherein X₈ is T or A, wherein X₉ is S or R, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

In some embodiments, piggy Bac transposase has the amino acid sequence: MSSLASNRX₁₀LTLPQRTX₁₁RGKNKHCWSTSKX₁₂TRRSRVSALX₁₃IVRSQRGPTRMCRNIYDP LLCFKLFFTDEIISEIVKWTNAEISLKRRESMTX₁₄ATFRDTNEDEIYAFFGILVX₁₅TAVRKDNHX₁₆S TDDLFDRSLSMVYVSVMSRDRFDFLIRCLRMDDKX₁₇IRPTLRENDVFTPVRKIWDLFIHQCIQN YTPGAHLTIDEQLLGFRGRCPFX₁₈X₁₉YIPNKPSKYGIKILMMCDSGTKYMINGMPYLGRX₂₀TQT NGVPLGEYYVKELSKPVHGSCRNITCDNWFTSIPLAKNLLQEPYKLTIVGTVRSNKREIPEVLKN SRSRPVGTSMFCFDGPLTLVSYKPKX₂₁AKMVYLLSSCDEDASX₂₂NESTGKPQMVMYYNQTKG GVDTLDQMCSVMTCSRKTNRWPMALLYGMINIACINSFIIYSHNVSSKGEKVX₂₃SRKX₂₄FMRX₂₅ LYMX₂₆LTSSFMRKRLEAPTLKRYLRDNISNILPX₂₇EVPGTSDDSTEEPVMKKRTYCTYCPSX₂₈I RRKAX₂₉AX₃₀CX₃₁KCKKVICREHNIDMCX₃₂SCX₃₃GTSDDSTEEPVMKKRTYCTYCPSX₂₈IRRKA X₂₉AX₃₀CX₃₁KCKKVICREHNIDMCX₃₂SCX₃₃ (SEQ ID NO:13), wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

As disclosed herein, deletion of the N-terminal 104 amino acids (Δ1-104PB) results in a transposase capable of LE/LE excision without integration. This can be used in systems where it is desirable to excise a nucleic acid sequence efficiently without subsequent integration. Therefore, one can use the Δ1-104PB to re-excise and remove an LE-LE transposon from the genome. In some embodiments, one could use Δ1-104PB almost like cre-lox to remove transposons of interest so as to not integrate or “hop” anywhere else.

Therefore, in some embodiments, the disclosed transposase lacks 75 or more of the N-terminal 104 amino acids. Therefore, in some embodiments, the disclosed piggyBac transposase has the amino acid sequence: X_{c}-RRSRVSALNIVRSQRGPTRMCRNIYDPLLCFKLFFTDEIISEIVKWTNAEISLKRRESMTGAT FRDTNEDEIYAFFGILVMTAVRKDNHMSTDDLFDRSLSMVYVSVMSRDRFDFLIRCLRMDDKSI RPTLRENDVFTPVRKIWDLFIHQCIQNYTPGAHLTIDEQLLGFRGRCPFRMYIPNKPSKYGIKILM MCDSGTKYMINGMPYLGRGTQTNGVPLGEYYVKELSKPVHGSCRNITCDNWFTSIPLAKNLLQ EPYKLTIVGTVRSNKREIPEVLKNSRSRPVGTSMFCFDGPLTLVSYKPKPAKMVYLLSSCDEDA SINESTGKPQMVMYYNQTKGGVDTLDQMCSVMTCSRKTNRWPMALLYGMINIACINSFIIYSHN VSSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCF (SEQ ID NO:14), wherein X_{c} is 75 to 104 aa of the amino acid sequence:

(SEQ ID NO: 15)
MGSSLDDEHILSALLQSDDELVGEDSDSEISDHVSEDDVQSDTEEAFID
EVHEVQPTSSGSEILDEQNVIEQPGSSLASNRILTLPQRTIRGKNKHCW
STSKST.

Therefore, in some embodiments, the disclosed piggy Bac transposase has the amino acid sequence: RRSRVSALNIVRSQRGPTRMCRNIYDPLLCFKLFFTDEIISEIVKWTNAEISLKRRESMTGATFR DTNEDEIYAFFGILVMTAVRKDNHMSTDDLFDRSLSMVYVSVMSRDRFDFLIRCLRMDDKSIRP TLRENDVFTPVRKIWDLFIHQCIQNYTPGAHLTIDEQLLGFRGRCPFRMYIPNKPSKYGIKILMM CDSGTKYMINGMPYLGRGTQTNGVPLGEYYVKELSKPVHGSCRNITCDNWFTSIPLAKNLLQE PYKLTIVGTVRSNKREIPEVLKNSRSRPVGTSMFCFDGPLTLVSYKPKPAKMVYLLSSCDEDASI NESTGKPQMVMYYNQTKGGVDTLDQMCSVMTCSRKTNRWPMALLYGMINIACINSFIIYSHNV SSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCF (SEQ ID NO:16), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:16.

In some embodiments, the transposase also has a tandem additional C-terminal domain. Therefore, in some embodiments, the disclosed piggy Bac transposase has the amino acid sequence: X_{c}—RRSRVSALNIVRSQRGPTRMCRNIYDPLLCFKLFFTDEIISEIVKWTNAEISLKRRESMTGAT FRDTNEDEIYAFFGILVMTAVRKDNHMSTDDLFDRSLSMVYVSVMSRDRFDFLIRCLRMDDKSI RPTLRENDVFTPVRKIWDLFIHQCIQNYTPGAHLTIDEQLLGFRGRCPFRMYIPNKPSKYGIKILM MCDSGTKYMINGMPYLGRGTQTNGVPLGEYYVKELSKPVHGSCRNITCDNWFTSIPLAKNLLQ EPYKLTIVGTVRSNKREIPEVLKNSRSRPVGTSMFCFDGPLTLVSYKPKPAKMVYLLSSCDEDA SINESTGKPQMVMYYNQTKGGVDTLDQMCSVMTCSRKTNRWPMALLYGMINIACINSFIIYSHN VSSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCF (SEQ ID NO:17)-X₀-X_{b}, wherein X_{c} is 75 to 104 aa of the amino acid sequence: MGSSLDDEHILSALLQSDDELVGEDSDSEISDHVSEDDVQSDTEEAFIDEVHEVQPTSSGSEIL DEQNVIEQPGSSLASNRILTLPQRTIRGKNKHCWSTSKST (SEQ ID NO:15), wherein X_{b} is 40-53 aa of the amino acid sequence GTSDDSTEEPVMKKRTYCTYCPSKIRRKANASCKKCKKVICREHNIDMCQSCF (SEQ ID NO: 5), and wherein X₀ is a linker comprising 0-20 amino acid residues.

Therefore, in some embodiments, the disclosed piggy Bac transposase has the amino acid sequence: RRSRVSALNIVRSQRGPTRMCRNIYDPLLCFKLFFTDEIISEIVKWTNAEISLKRRESMTGATFR DTNEDEIYAFFGILVMTAVRKDNHMSTDDLFDRSLSMVYVSVMSRDRFDFLIRCLRMDDKSIRP TLRENDVFTPVRKIWDLFIHQCIQNYTPGAHLTIDEQLLGFRGRCPFRMYIPNKPSKYGIKILMM CDSGTKYMINGMPYLGRGTQTNGVPLGEYYVKELSKPVHGSCRNITCDNWFTSIPLAKNLLQE PYKLTIVGTVRSNKREIPEVLKNSRSRPVGTSMFCFDGPLTLVSYKPKPAKMVYLLSSCDEDASI NESTGKPQMVMYYNQTKGGVDTLDQMCSVMTCSRKTNRWPMALLYGMINIACINSFIIYSHNV SSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCFGTSDDSTEEPVMKKRTYCTYC PSKIRRKANASCKKCKKVICREHNIDMCQSCF (SEQ ID NO:18), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:18.

In some embodiments, the transposase also has an N-terminal HA tag, such as MAYPYDVPDYATS (SEQ ID NO:19).

Therefore, in some embodiments, the disclosed transposase lacks 75 or more of the N-terminal 104 amino acids. Therefore, in some embodiments, the disclosed piggy Bac transposase has the amino acid sequence: MAYPYDVPDYATS (SEQ ID NO:19)-X_{c}-RRSRVSALNIVRSQRGPTRMCRNIYDPLLCFKLFFTDEIISEIVKWTNAEISLKRRESMTGATFR DTNEDEIYAFFGILVMTAVRKDNHMSTDDLFDRSLSMVYVSVMSRDRFDFLIRCLRMDDKSIRP TLRENDVFTPVRKIWDLFIHQCIQNYTPGAHLTIDEQLLGFRGRCPFRMYIPNKPSKYGIKILMM CDSGTKYMINGMPYLGRGTQTNGVPLGEYYVKELSKPVHGSCRNITCDNWFTSIPLAKNLLQE PYKLTIVGTVRSNKREIPEVLKNSRSRPVGTSMFCFDGPLTLVSYKPKPAKMVYLLSSCDEDASI NESTGKPQMVMYYNQTKGGVDTLDQMCSVMTCSRKTNRWPMALLYGMINIACINSFIIYSHNV SSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCF (SEQ ID NO:20), wherein X_{c} is 75 to 104 aa of the amino acid sequence:

(SEQ ID NO: 15)
MGSSLDDEHILSALLQSDDELVGEDSDSEISDHVSEDDVQSDTEEAFID
EVHEVQPTSSGSEILDEQNVIEQPGSSLASNRILTLPQRTIRGKNKHCW
STSKST.

Therefore, in some embodiments, the disclosed piggyBac transposase has the amino acid sequence: MAYPYDVPDYATSRRSRVSALNIVRSQRGPTRMCRNIYDPLLCFKLFFTDEIISEIVKWTNAEISL KRRESMTGATFRDTNEDEIYAFFGILVMTAVRKDNHMSTDDLFDRSLSMVYVSVMSRDRFDFU RCLRMDDKSIRPTLRENDVFTPVRKIWDLFIHQCIQNYTPGAHLTIDEQLLGFRGRCPFRMYIPN KPSKYGIKILMMCDSGTKYMINGMPYLGRGTQTNGVPLGEYYVKELSKPVHGSCRNITCDNWF TSIPLAKNLLQEPYKLTIVGTVRSNKREIPEVLKNSRSRPVGTSMFCFDGPLTLVSYKPKPAKMV YLLSSCDEDASINESTGKPQMVMYYNQTKGGVDTLDQMCSVMTCSRKTNRWPMALLYGMINI ACINSFIIYSHNVSSKGEKVQSRKKFMRNLYMSLTSSFMRKRLEAPTLKRYLRDNISNILPNEVP GTSDDSTEEPVMKKRTYCTYCPSKIRRKANASCKKCKKVICREHNIDMCQSCF (SEQ ID NO: 21), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:21.

In some embodiments, the transposase also has a tandem additional C-terminal domain. Therefore, in some embodiments, the disclosed piggy Bac transposase has the amino acid sequence: MAYPYDVPDYATS (SEQ ID NO: 19)-X_{c}-RRSRVSALNIVRSQRGPTRMCRNIYDPLLCFKLFFTDEIISEIVKWTNAEISLKRRESMTGATFR DTNEDEIYAFFGILVMTAVRKDNHMSTDDLFDRSLSMVYVSVMSRDRFDFLIRCLRMDDKSIRP TLRENDVFTPVRKIWDLFIHQCIQNYTPGAHLTIDEQLLGFRGRCPFRMYIPNKPSKYGIKILMM CDSGTKYMINGMPYLGRGTQTNGVPLGEYYVKELSKPVHGSCRNITCDNWFTSIPLAKNLLQE PYKLTIVGTVRSNKREIPEVLKNSRSRPVGTSMFCFDGPLTLVSYKPKPAKMVYLLSSCDEDASI NESTGKPQMVMYYNQTKGGVDTLDQMCSVMTCSRKTNRWPMALLYGMINIACINSFIIYSHNV SSKGEKVQSRKKFMRNLYMSLTSSFMRKRLEAPTLKRYLRDNISNILPNEVPGTSDDSTEEPV MKKRTYCTYCPSKIRRKANASCKKCKKVICREHNIDMCQSCF (SEQ ID NO:22)-X₀-X_{b}, wherein X_{c} is 75 to 104 aa of the amino acid sequence: MGSSLDDEHILSALLQSDDELVGEDSDSEISDHVSEDDVQSDTEEAFIDEVHEVQPTSSGSEIL DEQNVIEQPGSSLASNRILTLPQRTIRGKNKHCWSTSKST (SEQ ID NO:15), wherein X_{b} is 40-53 aa of the amino acid sequence GTSDDSTEEPVMKKRTYCTYCPSKIRRKANASCKKCKKVICREHNIDMCQSCF (SEQ ID NO: 6), and wherein X₀ is a linker comprising 0-20 amino acid residues.

Therefore, in some embodiments, the disclosed piggy Bac transposase has the amino acid sequence: MAYPYDVPDYATSRRSRVSALNIVRSQRGPTRMCRNIYDPLLCFKLFFTDEIISEIVKWTNAEISL KRRESMTGATFRDTNEDEIYAFFGILVMTAVRKDNHMSTDDLFDRSLSMVYVSVMSRDRFDFU RCLRMDDKSIRPTLRENDVFTPVRKIWDLFIHQCIQNYTPGAHLTIDEQLLGFRGRCPFRMYIPN KPSKYGIKILMMCDSGTKYMINGMPYLGRGTQTNGVPLGEYYVKELSKPVHGSCRNITCDNWF TSIPLAKNLLQEPYKLTIVGTVRSNKREIPEVLKNSRSRPVGTSMFCFDGPLTLVSYKPKPAKMV YLLSSCDEDASINESTGKPQMVMYYNQTKGGVDTLDQMCSVMTCSRKTNRWPMALLYGMINI ACINSFIIYSHNVSSKGEKVQSRKKFMRNLYMSLTSSFMRKRLEAPTLKRYLRDNISNILPNEVP GTSDDSTEEPVMKKRTYCTYCPSKIRRKANASCKKCKKVICREHNIDMCQSCFGTSDDSTEEP VMKKRTYCTYCPSKIRRKANASCKKCKKVICREHNIDMCQSCF (SEQ ID NO:23), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:23.

Disclosed here are modified RE and LE configurations of the Myotis lucifugus piggy Bat transposon with a truncated LE at 88 nucleotides from the LE cleavage site and 100 nucleotides from the RE cleavage site of the transposon. Without wishing to be bound by theory, this transposon end configuration results in substantially higher transposition activity in cells especially when these modified ends are used with a modified piggy Bat transposase.

In some embodiments, the core piggy Bac transposase is the piggy Bat transposase, such as a transposase described in WO 2010/085699, which is incorporated by reference for the teaching of these transposases and transposons. For example, in some embodiments, core piggy Bat transposase has the amino acid sequence: MSQHSDYSDDEFCADKLSNYSCDSDLENASTSDEDSSDDEVMVRPRTLRRRR ISSSSSDSESDIEGGREEWSHVDNPPVLEDFLGHQGLNTDAVINNIEDAVKLFIGDDFFEFLVEE SNRYYNQNRNNFKLSKKSLKWKDITPQEMKKFLGLIVLMGQVRKDRRDDYWTTEPWTETPYF GKTMTRDRFRQIWKAWHFNNNADIVNESDRLCKVRPVLDYFVPKFINIYKPHQQLSLDEGIVPW RGRLFFRVYNAGKIVKYGILVRLLCESDTGYICNMEIYCGEGKRLLETIQTVVSPYTDSWYHIYM DNYYNSVANCEALMKNKFRICGTIRKNRGIPKDFQTISLKKGETKFIRKNDILLQVWQSKKPVYLI SSIHSAEMEESQNIDRTSKKKIVKPNALIDYNKHMKGVDRADQYLSYYSILRRTVKWTKRLAMY MINCALFNSYAVYKSVRQRKMGFKMFLKQTAIHWLTDDIPEDMDIVPDLQPVPSTSGMRAKPP TSDPPCRLSMDMRKHTLQAIVGSGKKKNILRRCRVCSVHKLRSETRYMCKFCNIPLHKGACFE KYHTLKNY (SEQ ID NO:24). In some embodiments, the underlined N-terminal 40 amino acids are may be unnecessary for LE:LE binding, and the underlined C-terminal 80 amino acids may be involved in LE:LE binding.

Therefore, in some embodiments, the disclosed piggyBat transposase has the amino acid sequence:

(SEQ ID NO: 25)
VMVRPRTLRRRRISSSSSDSESDIEGGREEWSHVDNPPVLEDFLGHQGL
NTDAVINNIEDAVKLFIGDDFFEFLVEESNRYYNQNRNNFKLSKKSLKW
KDITPQEMKKFLGLIVLMGQVRKDRRDDYWTTEPWTETPYFGKTMTRDR
FRQIWKAWHENNNADIVNESDRLCKVRPVLDYFVPKFINIYKPHQQLSL
DEGIVPWRGRLFFRVYNAGKIVKYGILVRLLCESDTGYICNMEIYCGEG
KRLLETIQTVVSPYTDSWYHIYMDNYYNSVANCEALMKNKFRICGTIRK
NRGIPKDFQTISLKKGETKFIRKNDILLQVWQSKKPVYLISSIHSAEME
ESQNIDRTSKKKIVKPNALIDYNKHMKGVDRADQYLSYYSILRRTVKWT
KRLAMYMINCALFNSYAVYKSVRQRKMGFKMFLKQTAIHWLTDDIPEDM
DIVPDLQPVPSTSGMRAKPPTSDPPCRLSMDMRKHTLQAIVGSGKKKNI
LRRCRVCSVHKLRSETRYMCKFCNIPLHKGACFEKYHTLKNY.

Therefore, in some embodiments, the disclosed piggy Bat transposase has the amino acid sequence:

(SEQ ID NO: 26)
MSQHSDYSDDEFCADKLSNYSCDSDLENASTSDEDSSDDEVMVRPRTLR
RRRISSSSSDSESDIEGGREEWSHVDNPPVLEDFLGHQGLNTDAVINNI
EDAVKLFIGDDFFEFLVEESNRYYNQNRNNFKLSKKSLKWKDITPQEMK
KFLGLIVLMGQVRKDRRDDYWTTEPWTETPYFGKTMTRDRFRQIWKAWH
ENNNADIVNESDRLCKVRPVLDYFVPKFINIYKPHQQLSLDEGIVPWRG
RLFFRVYNAGKIVKYGILVRLLCESDTGYICNMEIYCGEGKRLLETIQT
WSPYTDSWYHIYMDNYYNSVANCEALMKNKFRICGTIRKNRGIPKDFQT
ISLKKGETKFIRKNDILLQVWQSKKPVYLISSIHSAEMEESQNIDRTSK
KKIVKPNALIDYNKHMKGVDRADQYLSYYSILRRTVKWTKRLAMYMINC
ALFNSYAVYKSVRQRKMGFKMFLKQTAIHWLTDDIPEDMDIVPDLQPVP
STSGMRAKPPTSDPPCRLSMDMRKHTLQAIVGSGKKKNILRRCRVCSVH
KLRSETRYMCKFCNIPLHKGACFEKYHTLKNSTSGMRAKPPTSDPPCRL
SMDMRKHTLQAIVGSGKKKNILRRCRVCSVHKLRSETRYMCKFCNIPLH
KGACFEKYHTLKNY.

Therefore, in some embodiments, the disclosed piggyBat transposase has the amino acid sequence:

(SEQ ID NO: 27)
VMVRPRTLRRRRISSSSSDSESDIEGGREEWSHVDNPPVLEDFLGHQGL
NTDAVINNIEDAVKLFIGDDFFEFLVEESNRYYNQNRNNFKLSKKSLKW
KDITPQEMKKFLGLIVLMGQVRKDRRDDYWTTEPWTETPYFGKTMTRDR
FRQIWKAWHENNNADIVNESDRLCKVRPVLDYFVPKFINIYKPHQQLSL
DEGIVPWRGRLFFRVYNAGKIVKYGILVRLLCESDTGYICNMEIYCGEG
KRLLETIQTVVSPYTDSWYHIYMDNYYNSVANCEALMKNKFRICGTIRK
NRGIPKDFQTISLKKGETKFIRKNDILLQVWQSKKPVYLISSIHSAEME
ESQNIDRTSKKKIVKPNALIDYNKHMKGVDRADQYLSYYSILRRTVKWT
KRLAMYMINCALFNSYAVYKSVRQRKMGFKMFLKQTAIHWLTDDIPEDM
DIVPDLQPVPSTSGMRAKPPTSDPPCRLSMDMRKHTLQAIVGSGKKKNI
LRRCRVCSVHKLRSETRYMCKFCNIPLHKGACFEKYHTLKNSTSGMRAK
PPTSDPPCRLSMDMRKHTLQAIVGSGKKKNILRRCRVCSVHKLRSETRY
MCKFCNIPLHKGACFEKYHTLKNY.

Therefore, in some embodiments, the disclosed piggy Bat transposase has the amino acid sequence:

(SEQ ID NO: 33)
MSQHSDYADDEFCADKLSNYSCDADLENASTADEDSADDEVMVRPRTLR
RRRISSSSSDSESDIEGGREEWSHVDNPPVLEDFLGHQGLNTDAVINNI
EDAVKLFIGDDFFEFLVEESNRYYNQNRNNFKLSKKSLKWKDITPQEMK
KFLGLIVLMGQVRKDRRDDYWTTEPWTETPYFGKTMTRDRFRQIWKAWH
ENNNADIVNESDRLCKVRPVLDYFVPKFINIYKPHQQLSLDEGIVPWRG
RLFFRVYNAGKIVKYGILVRLLCESDTGYICNMEIYCGEGKRLLETIQT
WSPYTDSWYHIYMDNYYNSVANCEALMKNKFRICGTIRKNRGIPKDFQT
ISLKKGETKFIRKNDILLQVWQSKKPVYLISSIHSAEMEESQNIDRTSK
KKIVKPNALIDYNKHMKGVDRADQYLSYYSILRRTVKWTKRLAMYMINC
ALFNSYAVYKSVRQRKMGFKMFLKQTAIHWLTDDIPEDMDIVPDLQPVP
STSGMRAKPPTSDPPCRLSMDMRKHTLQAIVGSGKKKNILRRCRVCSVH
KLRSETRYMCKFCNIPLHKGACFEKYHTLKNSTSGMRAKPPTSDPPCRL
SMDMRKHTLQAIVGSGKKKNILRRCRVCSVHKLRSETRYMCKFCNIPLH
KGACFEKYHTLKNY.

In some embodiments, the disclosed piggyBac transposase has one or more hyperactive mutations described in U.S. Pat. No. 11,485,959, which is incorporated by reference in its entirety for the teaching of these mutations. In some embodiments, the disclosed piggyBac transposase has one or more mutations selected from the group consisting of S3N, I30V, A46S, A46T, I82W, S103P, R119P, C125A, C125L, G165S, Y177K, Y177H, F180L, F180I, F180V, M185L, A187G, F200W, V207P, V209F, M226F, L235R, V240K, F241L, P243K, N258S, M282Q, L296W, L296Y, L296F, M298L, M298A, M298V, P311I, P311V, R315K, T319G, Y327R, Y328V, C340G, C34L0, D421H, V436I, M456Y, L470F, S486K, M503L, M503I, V552K, A570T, Q591P, Q591R, or any combination thereof.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1E show piggyBac transposon organization, and identification of transposase N-terminal CKII dependent phosphorylation that inhibits activity in human cells. FIG. 1A is a schematic of PB transposon flanked by TTAA, and sequence and organization of the LE (SEQ ID NO:28) and RE (SEQ ID NO:29) TIRs. Internal repeat sequences are shown. FIG. 1B shows alignment of the N-terminus of piggyBac (SEQ ID NO:30) and piggyBat (SEQ ID NO: 31) transposases demonstrating CKII phosphorylation sites (highlighted). CKII sites within PB found to be phosphorylated when expressed in human cells are marked with a green box. FIG. 1C is a schematic of inter-plasmid in cell transposition assay. Isolated episomal DNA post-transfection was electroporated into bacteria which were plated on medium containing kanamycin to measure the total recovery of recipient plasmids or on kanamycin/tetracyline/streptomycin plates. Tet and Kan allow selection for the transposition product, and streptomycin selects against donor plasmids which contain the streptomycin sensitivity gene rpsL. FIG. 1D shows in cell transposition activity comparing phosphorylation site mutations to WT using a inter-plasmid transposition assay in human cells. FIG. 1 E shows colony count (integration) assay of a neomycin resistant transposon comparing AllStoA PB to WT PB in human cells. N=3±SEM; *, p<0.05 using student's T test.

FIGS. 2A to 2C show AlphaFold structural prediction for the piggy Bac transposase N-terminal region suggests multiple roles. FIGS. 2A and 2B show AlphaFold-Multimer modeling of full length piggyBac transposase suggests that N-terminus phosphorylation inhibits DNA binding. FIG. 2C shows absorbance sedimentation c(s) profiles for 5.8 μM PB1-558 and 5.4 μM PB74-539 show the presence of a dimer and monomer, respectively. Data collected for 1.4 μM PB4-539 showed a profile similar to that for the more concentrated sample.

FIGS. 3A to 3D show redesigned piggy Bac overcomes inhibition in human cells. FIG. 3A, left is a schematic of LE-RE vs LE-LE transposons containing a kanamycin/neomycin resistance cassette (Kan/NeoR) and a p15A origin of replication (p15A ori). FIG. 3A, right contains schematics of WT PB compared to Δ74PB and Δ74PB-2CD. The catalytic domain contains the conserved DDD motif. NTD N-terminal domain. Dimerization and DNA-binding domain (DDBD). CD, C-terminal cysteine-rich domain. FIG. 3B is a schematic of transposition in human cells evaluated via transposon excision and integration (colony count) assays. FIG. 3C shows excision assay analysis demonstrating ethidium bromide-stained gel of excision products of Δ74PB and Δ74PB-2CD with LE-LE compared to WT PB and hyPB with LE-RE. Shown is representative of three independent experiments. FIG. 3D shows colony count (integration) assay analysis of Δ74PB and Δ74PB-2CD with LE-LE compared to WT PB and hyPB with LE-RE. N=3±SEM; *, p<0.05 compared to PB or hyPB respectively with LE-RE using one way ANOVA and Turkey multiple comparisons test.

FIGS. 4A and 4B show Δ104PB is an excision active/integration inactive transposase on symmetric LE-LE TIRs. FIG. 4A shows excision assay analysis of Δ104PB compared to WT and Δ74PB in human cells. Shown is representative of 3 independent experiments. FIG. 4B shows colony count (integration) analysis of Δ104PB compared to WT and Δ74PB in human cells. N=3±SEM; *, p<0.05 using one way ANOVA and Dunnett's multiple comparisons test compared to no transposase control.

FIGS. 5A and 5B show structure-based redesign of piggy Bac for symmetric transposon ends. FIG. 5A shows structure of PB bound to LE-LE transposon. FIG. 5B is a model of asymmetric PB tetramer bound to LE-RE transposon. Re-design permits symmetric PB dimer to bind LE-LE transposon via appending a 2CD to the end of the PB transposase.

FIGS. 6A to 6C show redesigned piggy Bac overcomes inhibition over a range of transposase doses in human cells. FIG. 6A shows excision assay analysis of Δ74PB and Δ74PB-2CD with LE-LE compared to WT PB and hyPB with LE-RE over a range of transposase dosages while keeping transposon DNA constant at 1.5 μg. Shown is representative of 3 independent experiments. FIG. 6B shows colony count (integration) analysis corresponding to the excision analysis in a. N=3±SEM; *, p<0.05 comparing Δ74PB and Δ74PB-2CD with LE-LE to PB or hyPB respectively with LE-RE using one way ANOVA and Šídak's multiple comparisons test. FIG. 6C shows ddPCR copy number analysis of the number of integrated transposons in human cells normalized for the RNaseP gene. N=2±SEM.

FIGS. 7A and 7B show redesigned piggy Bac overcomes inhibition over a range of transposon sizes in human cells. FIG. 7A shows schematics of transposon vectors of varying sizes ranging from 3.4 to 15.1 kb. FIG. 7B shows colony count (integration) analysis corresponding to transposon sizes in FIG. 7A. N=3±SEM; *, p<0.05 compared to PB or hyPB respectively with LE-RE using one way ANOVA and Turkey multiple comparisons test using the transposons in FIG. 7A.

FIGS. 8A to 8D show genome-wide characterization of insertions sites by redesigned transposomes. FIG. 8A shows the sequence logo of the 5′ insertion site (first 15 nucleotides, SEQ ID NO:32) including the transposon inverted repeat (TIR) and the target site showed consistent excision and target site duplication (TSD) for wild-type PB, hyperactive mutants, and different TIRs. Three biological replicates were analyzed, and a single representative replicate is shown. FIG. 8B shows the overall genome-wide distribution of insertion-peaks remained unchanged between wild-type and mutant transposomes across different chromosomes [mean value, N=3]. FIG. 8C shows annotation of insertion-peaks by genomic features showed comparable preferences for different genomic regions [mean±SEM, N=3]. Genic versus intergenic regions, protein coding genes, and protein coding exons are depicted. The genomic contribution of these regions is indicated as pie charts for comparison. FIG. 8D shows insertions-peaks for wild type and mutant piggy Bac transposomes are shown for a representative genomic region with high-density insertions on Chromosome 7. Insertion densities are shown as normalized read coverage (rpm), and peaks are indicated.

FIG. 9 shows the schematic and sequences of active piggy Bat transposon ends. A top is a schematic of piggy Bat transposon as isolated from M. lucifugus. LE: Left End. RE: Right End. In the middle is a schematic of piggyBat transposon used for in cell culture assays. At bottom are sequences of active piggy Bat transposon ends (SEQ ID NOs: 34 and 35) showing two repeated motifs identified on the LE.

FIGS. 10A and B demonstrate that mutations of putative phosphorylation sites on the N-terminal domain show hyperactivity in cells. FIG. 10A shows alignment of piggy Bat (SEQ ID NO: 36) and piggy Bac (SEQ ID NO:37) N-termini highlighting the CK II SDXD/E phosphorylation motifs (underlined with bold indicating the putative phosphorylated serine residue). FIG. 10B shows the colony counts for the piggy Bat transposase and variants with various truncated LE and RE. Numbers indicated how long the ends are from the TTAA transposase cut site.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the term “polypeptide” is meant to refer to a polymer of amino acids of any length. Thus, for example, the terms peptide, oligopeptide, protein, antibody, and enzyme are included within the definition of polypeptide. This term also includes post-expression modifications of the polypeptide, for example, glycosylations (e.g., the addition of a saccharide), acetylations, phosphorylations and the like.

As used herein, the term “transposon” or “transposable element” is meant to refer to a polynucleotide that is able to excise from a donor polynucleotide, for instance, a vector, and integrate into a target site, for instance, a cell's genomic or extrachromosomal DNA. A transposon includes a polynucleotide that includes a nucleic acid sequence flanked by cis-acting nucleotide sequences on the termini of the transposon. A nucleic acid sequence is “flanked by” cis-acting nucleotide sequences if at least one cis-acting nucleotide sequence is positioned 5′ to the nucleic acid sequence, and at least one cis-acting nucleotide sequence is positioned 3′ to the nucleic acid sequence. Cis-acting nucleotide sequences include at least one inverted repeat (also referred to herein as an terminal inverted repeat, or TIR) at each end of the transposon, to which a transposase, preferably a member of the piggy Bac family of transposases, binds. In certain preferred embodiments, the transposon is from the family Noctuidae. In further preferred embodiments, the transposon is a Trichoplusia ni (Cabbage looper moth) piggyBac transposon or the Myotis lucifugus Piggy Bat transposon.

As used herein “Trichoplusia ni” is meant to refer to a member of the moth family Noctuidae.

An “isolated” polypeptide or polynucleotide means a polypeptide or polynucleotide that has been either removed from its natural environment, produced using recombinant techniques, or chemically or enzymatically synthesized. Preferably, a polypeptide or polynucleotide of this invention is purified, i.e., essentially free from any other polypeptide or polynucleotide and associated cellular products or other impurities.

As used herein, the term “transposase” is meant to refer to a polypeptide that catalyzes the excision of a transposon from a donor polynucleotide (e.g., a vector) and the subsequent integration of the transposon into the genomic or extrachromosomal DNA of a target cell. Preferably, the transposase binds an inverted sequence or a direct repeat. The transposase may be present as a polypeptide. Alternatively, the transposase is present as a polynucleotide that includes a coding sequence encoding a transposase. The polynucleotide can be RNA, for instance an mRNA encoding the transposase, or DNA, for instance a coding sequence encoding the transposase. When the transposase is present as a coding sequence encoding the transposase, in some aspects of the invention the coding sequence may be present on the same vector that includes the transposon, i.e., in cis. In other aspects of the invention, the transposase coding sequence may be present on a second vector, i.e., in trans. In certain preferred embodiments, the transposase is a mammalian piggyBac transposase.

Assays for measuring the excision of a transposon from a vector, the integration of a transposon into the genomic or extrachromosomal DNA of a cell, and the ability of transposase to bind to an inverted repeat are described herein and are known to the art (see, for instance, (Ivics et al. Cell, 91, 501-510 (1997); WO 98/40510 (Hackett et al.); WO 99/25817 (Hackett et al.), WO 00/68399 (McIvor et al.), incorporated by reference in their entireties herein. For purposes of determining the frequency of transposition of a transposon of the present invention, the activity of the baseline transposon is normalized to 100%, and the relative activity of the transposon of the present invention determined. Preferably, a transposon of the present invention transposes at a frequency that is, in increasing order of preference, at least about 50%, at least about 100%, at least about 200%, most preferably, at least about 300% greater than a baseline transposon. Preferably, both transposons (i.e., the baseline transposon and the transposon being tested) are flanked by the same nucleotide sequence in the vector containing the transposons.

Amino acid substitutions as described herein are substitutions that enhance the transposition activity of the resulting transposase.

Amino acid insertions and substitutions are preferably carried out at those sequence positions of that do not alter the spatial structure or which relate to the catalytic center or binding region of the piggyBac transposon or transposase. A change of a spatial structure by insertion(s) or deletion(s) can be detected readily with the aid of, for example, CD spectra (circular dichroism spectra) (Urry, 1985, Absorption, circular Dichroism and ORD of Polypeptides, in: Modern Physical Methods in Biochemistry, Neuberger et al. (Ed.), Elsevier, Amsterdam). Suitable methods for generating proteins with amino acid sequences which contain substitutions in comparison with the native sequence(s) are disclosed for example in the publications U.S. Pat. Nos. 4,737,462, 4,588,585, 4,959,314, 5,116,943, 4,879,111 and 5,017,691, incorporated by reference in their entireties herein. Other functional derivatives may be additionally stabilized in order to avoid physiological degradation. Such stabilization may be obtained by stabilizing the protein backbone by a substitution of by stabilizing the protein backbone by substitution of the amide-type bond, for example also by employing [beta]-amino acids.

The disclosed piggy Bac transposase and piggy Bat transposase, in combination with the corresponding transposon as defined above can be transfected into a cell as a protein or as ribonucleic acid, including mRNA, as DNA, e.g. as extrachromosomal DNA including, but not limited to, episomal DNA, as plasmid DNA, or as viral nucleic acid. Furthermore, the nucleic acid encoding the transposase protein can be transfected into a cell as a nucleic acid vector such as a plasmid, or as a gene expression vector, including a viral vector. Therefore, the nucleic acid can be circular or linear. A vector, as used herein, refers to a plasmid, a viral vector or a cosmid that can incorporate nucleic acid encoding the transposase protein or the transposon of this invention. The terms “coding sequence” or “open reading frame” refer to a region of nucleic acid that can be transcribed and/or translated into a polypeptide in vivo when placed under the control of the appropriate regulatory sequences.

DNA encoding the transposase protein can be stably inserted into the genome of the cell or into a vector for constitutive or inducible expression. Where the transposase protein is transfected into the cell or inserted into the vector as nucleic acid, the transposase encoding sequence is preferably operably linked to a promoter. There are a variety of promoters that could be used including, but not limited to, constitutive promoters, tissue-specific promoters, inducible promoters, and the like. Promoters are regulatory signals that bind RNA polymerase in a cell to initiate transcription of a downstream (3′ direction) coding sequence. A DNA sequence is operably linked to an expression-control sequence, such as a promoter when the expression control sequence controls and regulates the transcription and translation of that DNA sequence. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. In addition to the conservative changes discussed above that would necessarily alter the transposon-encoding nucleic acid sequence (all of which are disclosed herein as well), there are other DNA or RNA sequences encoding the hyperactive piggyBac transposon protein. These DNA or RNA sequences have the same amino acid sequence as a hyperactive piggyBac transposon protein, but take advantage of the degeneracy of the three letter codons used to specify a particular amino acid. For example, it is well known in the art that various specific RNA codons (corresponding DNA codons, with a T substituted for a U) can be used interchangeably to code for specific amino acids.

Methods for manipulating DNA and proteins are known in the art and are explained in detail in the literature such as Sambrook et al, (1989) Molecular Cloning: A Laboratory Manual, Cold Spring Harbor Laboratory Press or Ausubel, R. M., ed. (1994). Current Protocols in Molecular Biology.

Also disclosed herein is a gene transfer system involving a transposon in an LE/LE configuration and a piggyBac transposase as described herein.

As mentioned above, the piggyBac transposase protein preferably recognizes inverted repeats (e.g. TIRs) at the ends of the hyperactive piggy Bac transposon. The gene transfer system therefore preferably comprises two components: the transposase as described herein and an LE/LE transposon as described herein. In certain embodiments, the transposon has at least two repeats (e.g. IRs). When put together these two components provide active transposon activity and allow the transposon to be relocated. In use, the transposase binds to the TIRs and promotes insertion of the intervening nucleic acid sequence into DNA of a cell as defined below.

As mentioned above, similarly to piggy Bac transposase, the piggy Bat transposase protein also preferably recognizes inverted repeats (e.g. TIRs). The hyperactive piggy Bat gene transfer system therefore preferably comprises two components: the transposase as described herein and a truncated LE that contains only the first 88 nucleotides of the transposon left end and only the first 100 nucleotides of the transposon Right End. When put together these two components provide active transposon activity and allow the transposon to be relocated. In use, the transposase binds to the truncated TIRs and promotes insertion of the intervening nucleic acid sequence into DNA of a cell as defined below.

In certain embodiments, the gene transfer system mediates insertion of a piggyBac transposon or piggy Bat transposon into the DNA of a variety of cell types and a variety of species by using the disclosed piggyBac or piggy Bat transposase protein. Preferably, such cells include any cell suitable in the present context, including but not limited to animal cells or cells from bacteria, fungi (e.g., yeast, etc.) or plants. Preferred animal cells can be vertebrate or invertebrate. For example, preferred vertebrate cells include cells from mammals including, but not limited to, rodents, such as rats or mice, ungulates, such as cows or goats, sheep, swine or cells from a human.

In other further exemplary embodiments, such cells, particularly cells derived from a mammals as defined above, can be pluripotent (i.e., a cell whose descendants can differentiate into several restricted cell types, such as hematopoietic stem cells or other stem cells) and totipotent cells (i.e., a cell whose descendants can become any cell type in an organism, e.g., embryonic stem cells). These cells are advantageously used in order to affirm stable expression of the transposase or to obtain a multiple number of cells already transfected with the components of the inventive gene transfer system. Additionally, cells such as oocytes, eggs, and one or more cells of an embryo may also be considered as targets for stable transfection with the present gene transfer system. In certain embodiments, the cells are stem cells.

Cells receiving the inventive piggy Bac or piggy Bat transposon and/or the corresponding piggy Bac or piggy Bat transposase protein and capable of inserting the transposon into the DNA of that cell also include without being limited thereto, lymphocytes, hepatocytes, neural cells, muscle cells, a variety of blood cells, and a variety of cells of an organism, embryonic stem cells, somatic stem cells e.g. hematopoietic cells, embryos, zygotes, sperm cells (some of which are open to be manipulated by an in vitro setting).

In other certain exemplary embodiments, the cell DNA that acts as a recipient of the transposon of described herein includes any DNA present in a cell (as mentioned above) to be transfected, if the piggy Bac (or piggy Bat) transposon is in contact with the disclosed piggyBac (or piggyBat) transposase protein within the cell. For example, the DNA can be part of the cell genome or it can be extrachromosomal, such as an episome, a plasmid, a circular or linear DNA fragment. Typical targets for insertion are e.g. double-stranded DNA.

The components of the gene transfer system described herein, i.e. the piggyBac (or piggyBat) transposase protein (either as a protein or encoded by a nucleic acid as described herein) and a piggyBac (or piggyBat) transposon can be transfected into a cell. Transfection of these components may furthermore occur in subsequent order or in parallel, e.g. the piggy Bac (or piggy Bat) transposase protein or its encoding nucleic acid may be transfected into a cell as defined above prior to, simultaneously with or subsequent to transfection of the mammalian piggyBac (or piggy Bat) transposon. Alternatively, the transposon may be transfected into a cell as defined above prior to, simultaneously with or subsequent to transfection of the piggy Bac transposase protein or its encoding nucleic acid. Additionally, administration of at least one component of the gene transfer system may occur repeatedly, e.g. by administering at least one, two or multiple doses of this component.

For any of the above transfection reactions, the gene transfer system may be formulated in a suitable manner as known in the art, or as a pharmaceutical composition or kit as described herein.

The components of the gene transfer system may be transfected into one or more cells by techniques such as particle bombardment, electroporation, microinjection, combining the components with lipid-containing vesicles, such as cationic lipid vesicles, DNA condensing reagents (e.g., calcium phosphate, polylysine or polyethyleneimine), and inserting the components (i.e. the nucleic acids thereof into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector.

As already mentioned above the nucleic acid encoding the piggy Bac (or piggyBat) transposase protein may be RNA or DNA. Similarly, either the nucleic acid encoding the piggy Bac transposase protein or the transposon of this invention can be transfected into the cell as a linear fragment or as a circularized fragment, such as a plasmid or as recombinant viral DNA.

Furthermore, the nucleic acid encoding the piggyBac (or piggy Bat) transposase protein is thereby stably or transiently inserted into the genome of the cell to facilitate temporary or prolonged expression of the piggyBac (or piggyBat) transposase protein in the cell.

The gene transfer system as disclosed above represents a considerable refinement of non-viral DNA-mediated gene transfer. For example, adapting viruses as agents for gene therapy restricts genetic design to the constraints of that virus genome in terms of size, structure and regulation of expression. Non-viral vectors, as described herein, are generated largely from synthetic starting materials and are therefore more easily manufactured than viral vectors. Non-viral reagents are less likely to be immunogenic than viral agents making repeat administration possible. Non-viral vectors are more stable than viral vectors and therefore better suited for pharmaceutical formulation and application than are viral vectors. Additionally, the inventive gene transfer system is a non-viral gene transfer system that facilitates insertion into DNA and markedly improves the frequency of stable gene transfer.

Also disclosed herein is an efficient method for producing transgenic animals, including the step of applying the gene transfer system to an animal. Transgenic DNA typically is not efficiently inserted into chromosomes. Only about one in a million of the foreign DNA molecules is inserted into the cellular genome, generally several cleavage cycles into development. Consequently, most transgenic animals are mosaic (Hackett et al. (1993). The molecular biology of transgenic fish. In Biochemistry and Molecular Biology of Fishes (Hochachka & Mommsen, eds) Vol. 2, pp. 207-240). As a result, animals raised from embryos into which transgenic DNA has been delivered must be cultured until gametes can be assayed for the presence of inserted foreign DNA. Many transgenic animals fail to express the transgene due to position effects. A simple, reliable procedure that directs early insertion of exogenous DNA into the chromosomes of animals at the one-cell stage is needed. The present system helps to fill this need.

In certain preferred embodiments, the gene transfer system can readily be used to produce transgenic animals that carry a particular marker or express a particular protein in one or more cells of the animal. Generally, methods for producing transgenic animals are known in the art and incorporation of the gene transfer system into these techniques does not require undue experimentation, e.g. there are a variety of methods for producing transgenic animals for research or for protein production including, but not limited to Hackett et al. (1993, supra). Other methods for producing transgenic animals are described in the art (e.g. M. Markkula et al. Rev. Reprod., 1, 97-106 (1996); R. T. Wall et al., J. Dairy Sci., 80, 2213-2224 (1997)), J. C. Dalton, et al. (Adv. Exp. Med. Biol., 411, 419-428 (1997)) and H. Lubon et al. (Transfus. Med. Rev., 10, 131-143 (1996)).

Transgenic animals may be selected from vertebrates and invertebrates, e.g. fish, birds, mammals including, but not limited to, rodents, such as rats or mice, ungulates, such as cows or goats, sheep, swine or humans.

Also disclosed herein is a method for gene therapy that involves the step of introducing the gene transfer system into cells as described herein. Therefore, the piggyBac and piggyBat transposons as described herein preferably comprises a gene to provide a gene therapy to a cell or an organism. Preferably, the gene is placed under the control of a tissue specific promoter or of a ubiquitous promoter or one or more other expression control regions for the expression of a gene in a cell in need of that gene. Presently, a variety of genes are being tested for a variety of gene therapies including, but not limited to, the CFTR gene for cystic fibrosis, adenosine deaminase (ADA) for immune system disorders, factor IX and interleukin-2 (IL-2) for blood cell diseases, alpha-1-antitrypsin for lung disease, and tumor necrosis factors (INFs) and multiple drug resistance (MDR) proteins for cancer therapies. These and a variety of human or animal specific gene sequences including gene sequences to encode marker proteins and a variety of recombinant proteins are available in the known gene databases such as GenBank.

An advantage of the disclosed gene transfer system for gene therapy purposes is that it is not limited to a great extent by the size of the intervening nucleic acid sequence positioned between the repeats. There is no known limit on the size of the nucleic acid sequence that can be inserted into DNA of a cell using the piggy Bac transposase or the mammalian piggy Bat protein.

The gene transfer system may be transfected into cells by a variety of methods, e.g. by microinjection, lipid-mediated strategies or by viral-mediated strategies. For example, where microinjection is used, there is very little restraint on the size of the intervening sequence of the transposon of this invention. Similarly, lipid-mediated strategies do not have substantial size limitations. However, other strategies for introducing the gene transfer system into a cell, such as viral-mediated strategies could limit the length of the nucleic acid sequence positioned between the repeats.

Accordingly, in certain exemplary embodiments, the gene transfer system as described herein can be delivered to cells via viruses, including retroviruses (such as lentiviruses, etc.), adenoviruses, adeno-associated viruses, herpes viruses, and others. There are several potential combinations of delivery mechanisms that are possible for the piggyBac (or piggy Bat) transposon portion containing the transgene of interest flanked by the terminal repeats and the gene encoding the transposase. For example, both the transposon and the transposase gene can be contained together on the same recombinant viral genome; a single infection delivers both parts of the gene transfer system such that expression of the transposase then directs cleavage of the transposon from the recombinant viral genome for subsequent insertion into a cellular chromosome. In another example, the transposase and the transposon can be delivered separately by a combination of viruses and/or non-viral systems such as lipid-containing reagents. In these cases either the transposon and/or the transposase gene can be delivered by a recombinant virus. In every case, the expressed transposase gene directs liberation of the transposon from its carrier DNA (viral genome) for insertion into chromosomal DNA.

In certain embodiments, piggy Bac and piggy Bat transposons may be utilized for insertional mutagenesis, preferably followed by identification of the mutated gene. DNA transposons have several advantages compared to approaches in the prior art, e.g. with respect to viral and retroviral methods. For example, unlike proviral insertions, transposon insertions can be remobilized by supplying the transposase activity in trans. Thus, instead of performing time-consuming microinjections, it is possible to generate transposon insertions at new loci by crossing stocks transgenic for the above mentioned two components of the transposon system, the transposon and disclosed transposase. In some embodiments, the gene transfer system is directed to the germline of the experimental animals in order to mutagenize germ cells. Alternatively, transposase expression can be directed to particular tissues or organs by using a variety of specific promoters. In addition, remobilization of a mutagenic transposon out of its insertion site can be used to isolate revertants and, if transposon excision is associated with a deletion of flanking DNA, the inventive gene transfer system may be used to generate deletion mutations. Furthermore, since transposons are composed of DNA, and can be maintained in simple plasmids, inventive transposons and particularly the use of the inventive gene transfer system is much safer and easier to work with than highly infectious retroviruses. The transposase activity can be supplied in the form of DNA, mRNA or protein as defined above in the desired experimental phase.

Also disclosed is an efficient system for gene discovery, e.g. genome mapping, by introducing a piggyBac transposon, as defined above into a gene using a gene transfer system as described herein. In one example, the piggy Bac transposon in combination with the disclosed piggyBac transposase protein or a nucleic acid encoding the piggy Bac transposase protein is transfected into a cell. In certain embodiments, the transposon preferably comprises a nucleic acid sequence positioned between at least two TIRs, wherein the repeats bind to the piggyBac transposase protein and wherein the transposon is inserted into the DNA of the cell in the presence of the piggy Bac transposase protein. In certain preferred embodiments, the nucleic acid sequence includes a marker protein, such as GFP and a restriction endonuclease recognition site. Following insertion, the cell DNA is isolated and digested with the restriction endonuclease. For example, if the endonuclease recognition site is a 6-base recognition site and a restriction endonuclease is used that employs a 6-base recognition sequence, the cell DNA is cut into about 4000-bp fragments on average. These fragments can be either cloned or linkers can be added to the ends of the digested fragments to provide complementary sequence for PCR primers. Where linkers are added, PCR reactions are used to amplify fragments using primers from the linkers and primers binding to the direct repeats of the repeats in the transposon. The amplified fragments are then sequenced and the DNA flanking the direct repeats is used to search computer databases such as GenBank.

Using the gene transfer system for methods as disclosed above such as gene discovery and/or gene tagging, permits, for example, identification, isolation, and characterization of genes involved with growth and development through the use of transposons as insertional mutagens or identification, isolation and characterization of transcriptional regulatory sequences controlling growth and development.

Also disclosed is a method for mobilizing a nucleic acid sequence in a cell. According to this method the piggy Bac (or piggy Bat) transposon is inserted into DNA of a cell, as described herein. The piggyBac (or piggyBat) transposase protein or nucleic acid encoding the piggy Bac transposase protein is transfected into the cell and the transposase protein is able to mobilize (i.e. move) the transposon from a first position within the DNA of the cell to a second position within the DNA of the cell. The DNA of the cell is preferably genomic DNA or extrachromosomal DNA. The method allows movement of the transposon from one location in the genome to another location in the genome, or for example, from a plasmid in a cell to the genome of that cell.

In another exemplary embodiments, the gene transfer system can also be used as part of a method involving RNA-interference techniques. RNA interference (RNAi), is a technique in which exogenous, double-stranded RNAs (dsRNAs), being complementary to mRNA's or genes/gene fragments of the cell, are introduced into this cell to specifically bind to a particular mRNA and/or a gene and thereby diminishing or abolishing gene expression. The technique has proven effective in Drosophila, Caenorhabditis elegans, plants, and recently, in mammalian cell cultures. In order to apply this technique in context with the present invention, the inventive transposon preferably contains short hairpin expression cassettes encoding small interfering RNAs (siRNAs), which are complementary to mRNA's and/or genes/gene fragments of the cell. These siRNAs have preferably a length of 20 to 30 nucleic acids, more preferably a length of 20 to 25 nucleic acids and most preferably a length of 21 to 23 nucleic acids. The siRNA may be directed to any mRNA and/or a gene, that encodes any protein as defined above, e.g. an oncogene. This use, particularly the use of mammalian piggyBac transposons for integration of siRNA vectors into the host genome provides a long-term expression of siRNA in vitro or in vivo and thus enables a long-term silencing of specific gene products.

Also disclosed herein are pharmaceutical compositions containing either a piggyBac transposase disclosed herein as a protein or encoded by a nucleic acid, or a gene transfer system as described herein comprising a piggyBac (or piggyBat) transposase as a protein or encoded by a nucleic acid, in combination with a piggy Bac (or piggyBat) transposon.

The pharmaceutical composition may optionally be provided together with a pharmaceutically acceptable carrier, adjuvant or vehicle. In this context, a pharmaceutically acceptable carrier, adjuvant, or vehicle according to the invention refers to a non-toxic carrier, adjuvant or vehicle that does not destroy the pharmacological activity of the component(s) with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, topically, rectally, nasally, buccally, vaginally or via an implanted reservoir.

The term parenteral as used herein includes subcutaneous, intravenous, intramuscular, intra-articular, intra-synovial, intrasternal, intrathecal, intrahepatic, intralesional and intracranial injection or infusion techniques. Preferably, the pharmaceutical compositions are administered orally, intraperitoneally or intravenously. Sterile injectable forms of the pharmaceutical compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions may be administered in the form of suppositories for rectal administration. These can be prepared by mixing the inventive gene transfer system or components thereof with a suitable non-irritating excipient that is solid at room temperature but liquid at rectal temperature and Therefore will melt in the rectum to release the drug. Such materials include cocoa butter, beeswax and polyethylene glycols.

The pharmaceutically acceptable compositions may also be administered topically, especially when the target of treatment includes areas or organs readily accessible by topical application, including diseases of the eye, the skin, or the lower intestinal tract. Suitable topical formulations are readily prepared for each of these areas or organs.

For topical applications, the pharmaceutically acceptable compositions may be formulated in a suitable ointment containing the inventive gene transfer system or components thereof suspended or dissolved in one or more carriers. Carriers for topical administration of the components of this invention include, but are not limited to, mineral oil, liquid petrolatum, white petrolatum, propylene glycol, polyoxyethylene, polyoxypropylene component, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the components that may be combined with the carrier materials to produce a composition in a single dosage form will vary depending upon the host treated, the particular mode of administration. It has to be noted that a specific dosage and treatment regimen for any particular patient will depend upon a variety of factors, including the activity of the specific component employed, the age, body weight, general health, sex, diet, time of administration, rate of excretion, drug combination, and the judgment of the treating physician and the severity of the particular disease being treated. The amount of a component of the present invention in the composition will also depend upon the particular component(s) in the composition.

The pharmaceutical composition is preferably suitable for the treatment of diseases, particular diseases caused by gene defects such as cystic fibrosis, hypercholesterolemia, hemophilia, immune deficiencies including HIV, Huntington disease, .-alpha.-anti-Trypsin deficiency, as well as cancer selected from colon cancer, melanomas, kidney cancer, lymphoma, acute myeloid leukemia (AML), acute lymphoid leukemia (ALL), chronic myeloid leukemia (CML), chronic lymphocytic leukemia (CLL), gastrointestinal tumors, lung cancer, gliomas, thyroid cancer, mamma carcinomas, prostate tumors, hepatomas, diverse virus-induced tumors such as e.g. papilloma virus induced carcinomas (e.g. cervix carcinoma), adeno carcinomas, herpes virus induced tumors (e.g. Burkitt's lymphoma, EBV induced B cell lymphoma), Hepatitis B induced tumors (Hepato cell carcinomas), HTLV-1 and HTLV-2 induced lymphoma, lung cancer, pharyngeal cancer, anal carcinoma, glioblastoma, lymphoma, rectum carcinoma, astrocytoma, brain tumors, stomach cancer, retinoblastoma, basalioma, brain metastases, medullo blastoma, vaginal cancer, pancreatic cancer, testis cancer, melanoma, bladder cancer, Hodgkin syndrome, meningeoma, Schneeberger's disease, bronchial carcinoma, pituitary cancer, mycosis fungoides, gullet cancer, breast cancer, neurinoma, spinalioma, Burkitt's lymphoma, laryngeal cancer, thymoma, corpus carcinoma, bone cancer, non-Hodgkin lymphoma, urethra cancer, CUP-syndrome, oligodendroglioma, vulva cancer, intestinal cancer, oesphagus carcinoma, small intestine tumors, craniopharyngeoma, ovarial carcinoma, ovarian cancer, liver cancer, leukemia, or cancers of the skin or the eye; etc.

Also disclosed herein are kits comprising a piggyBac (or piggyBat) transposase as a protein or encoded by a nucleic acid, and a piggyBac (or piggy Bat) transposon; or a gene transfer system as described herein comprising a piggyBac (or piggyNat) transposase as a protein or encoded by a nucleic acid as described herein, in combination with a piggyBac (or piggyBat) transposon; optionally together with a pharmaceutically acceptable carrier, adjuvant or vehicle, and optionally with instructions for use.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Transposase N-Terminal Phosphorylation and Asymmetric Transposon Ends Inhibit piggyBac Transposition in Mammalian Cells

### INTRODUCTION

The genomes of almost all organisms contain transposable elements that can move discrete pieces of DNA (called transposons) from one place to another or coordinate the generation of another transposon copy at a new location. Through this ability to rearrange DNA, transposons can contribute to adaptation, although they are usually considered neutral or detrimental to host species. While integration of a transposon into a new site brings the potential for adaptive rewiring of regulatory pathways, there is always the danger of inactivating important genes or inappropriately activating others. Thus, many transposons have evolutionarily drifted away from maximal activity, increasing their chances of co-existing within their hosts while maintaining some ability to remain mobile.

The inherent mobile properties of transposons have led to efforts to use them for genome engineering. They therefore complement other DNA-modifying systems that are being developed for gene targeting applications such as transcription activator-like effector (TALE) proteins or CRISPR-Cas (clustered regularly interspaced short palindromic repeats and associated protein)-based tools (Becker, S., et al. Gene and Genome Editing, 2021 2:100007; Boutin, J., et al. CRISPR J, 2022 5:19-30). Currently, the most widely used transposons for genome modification experiments in higher organisms are Sleeping Beauty (SB) (Ivics, Z., et al. Cell, 1997 91:501-510; Mates, L., et al. Nat Genet, 2009 41:753-761), a resurrected mariner transposon originally identified as inactive in fish, and piggyBac (Fraser, M. J., et al. Insect Mol. Biol., 1996 5:141-151; Elick, T. A., et al. Genetica, 1996 98:33-41; Ding, S., et al. Cell, 2005 122:473-483), an active transposon isolated from the cabbage looper moth. piggyBac exhibits unique properties compared to other DNA transposons including specificity for insertion at the tetranucleotide sequence TTAA and the advantageous ability to couple its genomic excision with seamless repair in the host cell (Fraser, M. J., et al. Insect Mol. Biol., 1996 5:141-151; Elick, T. A., et al. Genetica, 1996 98:33-41). piggyBac has been used for a wide range of biotechnology applications including generation of transgenic animals, functional genomics, cancer gene discovery, and cell and gene therapy (Ding, S., et al. Cell, 2005 122:473-483; Yusa, K., et al. Nat. Methods, 2009 6:363-369; Woltjen, K., et al. Nature, 2009 458:766-770; Rad, R., et al. Science, 2010 330:1104-1107; Kahlig, K. M., et al. Proc Natl Acad Sci USA, 2010 107:1343-1348; Madison, B. B., et al. Molecular Therapy-Nucleic Acids, 2022 29:979-995; Saito, S., et al. Blood, 2021 138:4813).

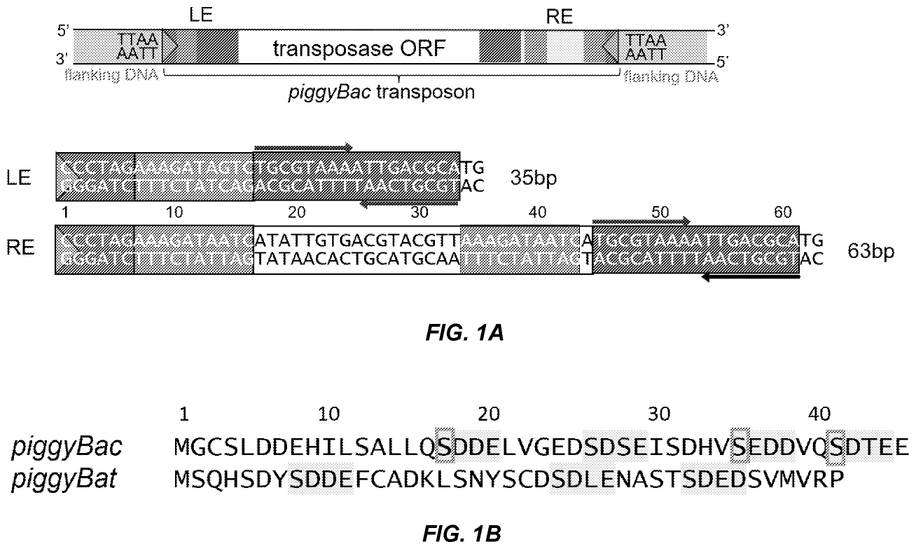

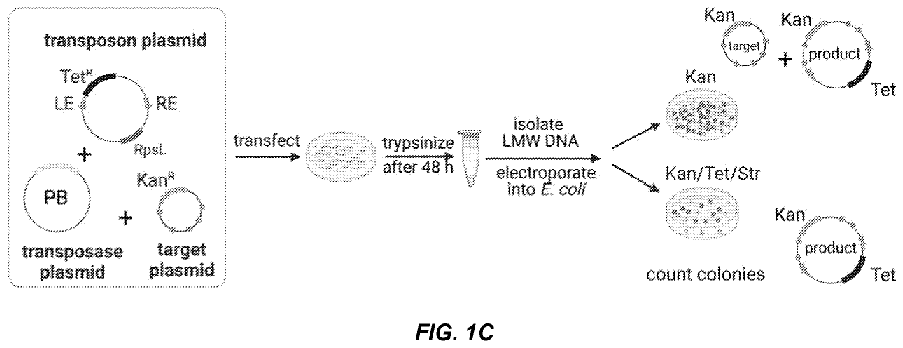

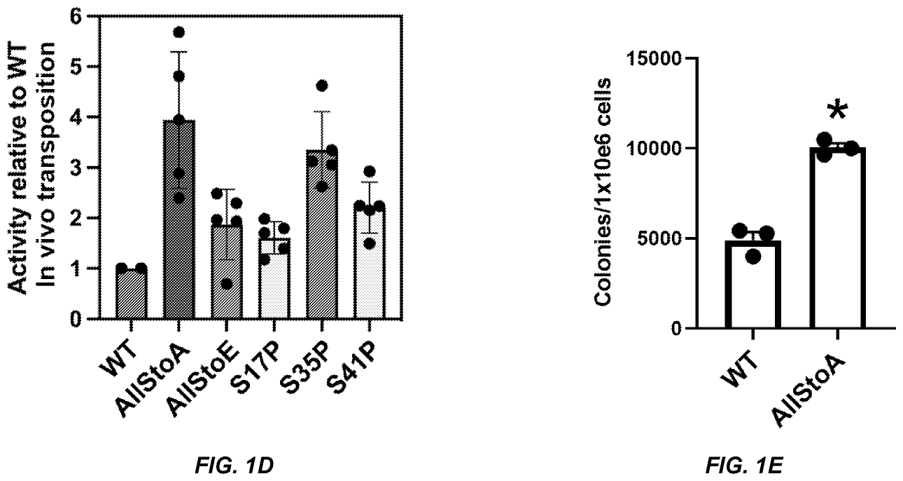

Although many properties of piggyBac have been uncovered and established (Gogol-Doring, A., et al. Molecular therapy 2016 24:592-606; Keith, J. H., et al. BMC molecular biology, 2008 9:72; Keith, J. H., et al. BMC molecular biology, 2008 9:73; Morellet, N., et al. Nucleic Acids Res, 2018 46:2660-2677; Li, X., et al. Proc Natl Acad Sci USA, 2013 110: E2279-2287; Mitra, R., et al. EMBO J., 2008 27:1097-1109), its further development for genomic applications would benefit from a deeper understanding not only of its mechanism of excision and integration, but also how its activity is regulated in mammalian cells. The wild-type (WT) piggyBac transposon consists of a single open reading frame encoding its transposase flanked by dissimilar transposon ends that contain several short DNA motifs arranged asymmetrically on its Left End (LE) and Right End (RE) (FIG. 1A). This asymmetry is crucial for transposition activity in cells (Chen, Q., et al. Nat Commun, 2020 11:3446). This remained a mystery until recent cryo-EM structures of the piggy Bac transposase bound to symmetric transposon hairpin ends and in a strand transfer complex with TTAA-containing target DNA provided evidence that the active assembly likely requires four transposase monomers (Chen, Q., et al. Nat Commun, 2020 11:3446). The structures provided valuable insights into the mechanistic details of the reactions carried out by the piggy Bac transposase yet raised new questions. For example, although the strand transfer transpososome (i.e., the complex of the transposase bound to DNA) contained its full complement of DNA substrates—two transposon ends and target DNA—the N-terminal region of the protein from residues 1-116 was unstructured and its role therefore remained unclear. A recent investigation showed that deletion of the first 100 amino acids abolished transposition activity (Wachtl, G., et al. Int. J. Mol. Sci., 202223:10317), a result that cannot be easily explained with current structural information.

How the activity of piggyBac might be regulated within mammalian cells is also not known. At the host level, the piggy Bac transposase has been shown to interact with DNA-dependent protein kinase which promotes pairing of the transposon ends (Jin, Y., et al. Proc Natl Acad Sci USA, 2017 114:7408-7413). The piggyBac transposase has also been reported to interact with bromodomain-containing proteins (i.e., BRD4) which appears to bias piggy Bac integration towards known sites of genomic DNA interaction with BRD4 (Gogol-Doring, A., et al. Molecular therapy 2016 24:592-606). As for the piggy Bac transposase, its activity appears to be constrained as demonstrated by the discovery of hyperactive piggyBac transposases generated via random mutagenesis of the transposase (Yusa, K., et al. Proc. Natl. Acad. Sci. U.S.A., 2011 108:1531-1536; Doherty, J. E., et al. Human Gene Therapy, 2012 23:311-320; Burnight, E. R., et al. Molecular therapy. Nucleic acids, 2012 1:e50). Transposition activity can also be increased by peptide addition to the transposase (Meir, Y. J., et al. FASEB journal 2013 27:4429-4443) or by manipulating the transposon ends (Hua, W.-K., et al. bioRxiv 2022). The current investigation was done to determine the previously unexplained functional role of the transposase N-terminus and to further investigate the need for asymmetric transposon ends and how this asymmetry may contribute to transposase tetramer formation. Another goal was to simplify the piggy Bac transpososome to advance its future capabilities for genome engineering.

### Materials and Methods

### Plasmid Constructs

pCMV-HAPB and pTpB with LE-RE and LE-LE TIRs have been described previously (Chen, Q., et al. Nat Commun, 2020 11:3446; Wilson, M. H., et al. Molecular Therapy, 2007 15:139-145). pCMV-hyPB has been described previously (Doherty, J. E., et al. Human Gene Therapy, 2012 23:311-320). pCMV-□74PB and pCMV-□74m7pB were generated by deleting N-terminal amino acids 1-74 using PCR while retaining an initiation methionine. pCMV-PB-2CD and pCMV-hyPB-2CD were generated by adding amino acids 542-594 of PB to the end of C-terminus in tandem. pCMV-□74PB-2CD and pCMV-□74hyPB-2CD were generated by deleting N-terminal amino acids 1-74 and adding amino acids 542-594 of PB to the end of C-terminus. pT-mAppleT2Apuro was generated by PCR amplifying the T2A peptide and puromycin resistance gene from PB-CMV-MCS-GreenPuro (System Biosciences, Cat #PB513B-1) and cloning into pT-mApple (Vectorbuilder). pT-mAppleT2Apuro-b-geo was generated by cloning the splice acceptor-b-geo fragment from PB-SB-SA-bgeo (Wang, W., et al. Proc. Natl. Acad. Sci. U.S.A., 2008 105:9290-9295) into pT-mAppleT2Apuro. pT-mAppleT2Apuro (15.1 kb) was generated by cloning a Pacl/BamHI restriction enzyme fragment from pAdEasy-1 (Addgene, Plasmid #16400) into pT-mApple. PB-SRT-Puro LE-RE and PB-SRT-Puro LE-LE were generated by replacing full length TIRs of PB-SRT-Puro (Moudgil, A., et al. Cell 2020 182:992-1008 e1021) using shorter LE and/or RE TIRs. Standard molecular biology techniques were used, and all constructs were confirmed with DNA sequencing.

For the plasmid-to-plasmid in cell assay, pFV4a-PB was synthesized by GenScript, and was derived from the Helraiser (HR) transposase expression plasmid, pFV4aRH (Grabundzija, I., et al. Nat Commun 2016 7:10716), by exchanging the HR ORF for that of human codon-optimized PB. pFV4a-PBAIIStoA, pFV4a-PBAlIStoE, pFV4a-PBS17P, pFV4a-PBS35P, and pFV4a-PBS41P were generated by ligation of the appropriate gBlock (IDT) between the SpeI and BmtI sites of pFV4a-PB. The donor plasmid, pTet-pBac-LE35-RE63, was synthesized by GenScript and was generated by replacing the 12- and 12-RSSs of pTet-RSS (Chatterji, M., et al. Mol Cell Biol 2006 26:1558-1568) with PB LE35 and RE63. pTet-RSS was a kind gift of the David Schatz lab and the target plasmid, pHSG298, was obtained from Takara Bio. pD2610-MBP-PB has been described previously (Yusa, K., et al. Proc. Natl. Acad. Sci. U.S.A., 2011 108:1531-1536). pD2610-MBP-PB1-539, pD2610-MBP-PB1-558, pD2610-MBP-TEVD74PB, and pD2610-MBP-TEVD74PB1-539 were synthesized by Twist Bioscience; for the TEVD74 constructs, the sequence ENLYFQG was inserted between amino acids G74 and S75.

### Protein Purification

PB1-539, TEVΔ74PB, and TEVΔ74PB1-539 were expressed in Expi293F cells (Thermo Fisher) and purified as previously described (Chen, Q., et al. Nat Commun, 2020 11:3446).

### Mass Spectrometry Analysis

Purified PB1-539 was run on a 4-12% NuPAGE gel and the corresponding band was excised for phosphorylation analysis by LC-MS/MS. The gel band was cut into ˜1 mm3 pieces, and the sample reduced with 1 mM DTT for 30 minutes at 60° C. followed by alkylation with 5 mM iodoacetamide for 15 minutes in the dark at room temperature. The sample was subjected to a modified in-gel AspN digestion procedure (Shevchenko, A., et al. Analytical Chemistry, 1996 68:850-858) as follows: Gel pieces were washed and dehydrated with acetonitrile for 10 min followed by removal of acetonitrile and then completely dried in a speed-vac. Rehydration was with 50 mM ammonium bicarbonate solution containing 12.5 ng/μl modified sequencing-grade AspN (ThermoScientific) at 4° C. The sample was then incubated at 37° C. overnight. Peptides were later extracted by removing the ammonium bicarbonate solution, followed by a wash with a 50% acetonitrile/1% formic acid solution. The extract was then dried in a speed-vac and stored at 4° C. until analysis.

For analysis, the sample was reconstituted in ˜10 μl of HPLC solvent A (2.5% acetonitrile, 0.1% formic acid). A nano-scale reverse-phase HPLC capillary column was created by packing 2.6 μm C18 spherical silica beads into a fused silica capillary (100 μm inner diameter x ˜30 cm length) with a flame-drawn tip (Peng, J., et al. J Mass Spectrom. 2001 36:1083-1091). After column equilibration, the sample was loaded onto the column via a Famos auto sampler (LC Packings, San Francisco CA). Peptides were eluted using a gradient with solvent B (97.5% acetonitrile, 0.1% formic acid).

As peptides were eluted, they were subjected to electrospray ionization and then they entered into an LTQ Orbitrap Velos Pro ion-trap mass spectrometer (Thermo Fisher Scientific, San Jose, CA). Eluting peptides were detected, isolated, and fragmented to produce a tandem mass spectrum of specific fragment ions for each peptide. Peptide sequences were determined by matching protein or translated nucleotide databases with the acquired fragmentation pattern by the software program, Sequest (ThermoFinnigan, San Jose, CA) (Eng, J. K., et al. J Am Soc Mass Spectrom 1994 5:976-989). The modification of 79.9663 mass units to serine, threonine, and tyrosine was included in the database searches to determine phosphopeptides. Phosphorylation assignments were determined by the Ascore algorithm (Beausoleil, S. A., et al. Nat Biotechnol, 2006 24:1285-1292). All databases include a reversed version of all the sequences and the data was filtered to between a 1-2% peptide false discovery rate.

### Sedimentation Velocity Analytical Ultracentrifugation (SV-AUC).

Purified PB1-558 in 500 mM NaCl, 20 mM HEPES·NaOH pH 7.6 and 0.5 mM TCEP, and PB74-539 in 500 mM NaCl, 25 mM TRIS·HCl PH 7.4 and 0.5 mM TCEP were prepared at ˜5. μM. Sedimentation velocity data were collected at 50,000 rpm and 20° C. on a Beckman Coulter ProteomeLab XL-I analytical ultracentrifuge following standard protocols (Zhao, H., et al. Curr Protoc Protein Sci, 2013 Chapter 20, Unit20 12). 2-channel, 12 mm centerpiece cells were used, and sedimentation was monitored with the absorbance (280 nm) and Rayleigh interference (655 nm) detection systems. A continuous c(s) distribution of Lamm equation solutions, as implemented in SEDFIT (Schuck, P. Biophys J 2000 78:1606-1619), modeled the sedimentation data. SEDNTERP (Cole, J. L., et al. Methods Cell Biol 2008 84:143-179) provided the solution densities ρ, solution viscosities η, and protein partial specific volumes required for the analysis.

### AlphaFold-Multimer

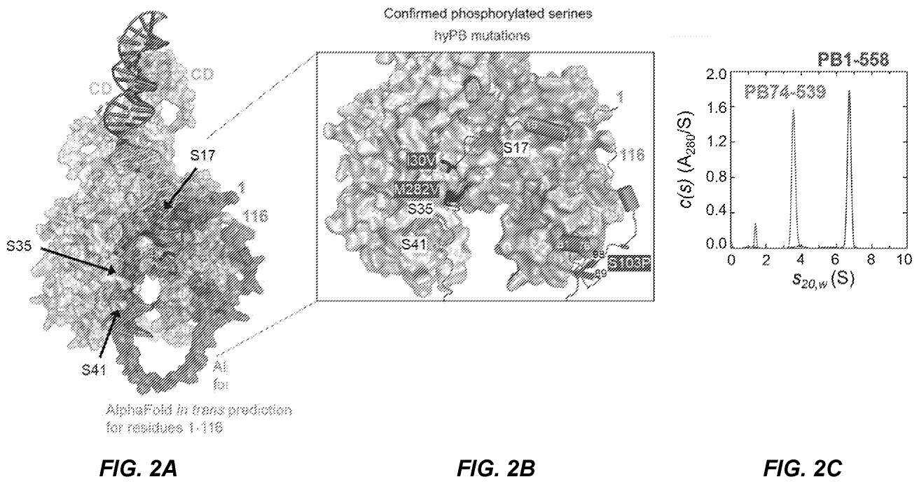

AlphaFold-Multimer (5×5) (Evans, R., et al. bioRxiv, 2022) was run on the NIH High Performance Computing system to generate 25 structural models for the full-length PB dimer from five different random seeds; the pLDDT scores ranged from 0.808 to 0.698. Among the ten models with the highest scores, three had regions in trans. Of these, the highest-ranking model is shown in FIG. 2A; two other models placed the first a-helix, residues 7-15, in trans.

### Cell Culture and Transfection

HT-1080 cells were cultured using standard procedures (Luo, W., et al. Nucleic Acids Res 2017 45:8411-8422). For transfection (unless otherwise indicated), cells were seeded at a density of 300,000 cells per well in a six-well plate and transfected with 2.5 μg of total plasmid DNA, containing 1.5 μg of transposon and 1 μg of transposase plasmid DNA unless otherwise indicated using Lipofectamine LTX (Invitrogen), according to manufacturer's instructions. For varying transposase DNA amount, cells were transfected with 1.5 μg of transposon and various transposase plasmid DNA amounts with pUC19 plasmid DNA added to make the total DNA amount 4 μg per condition. Cells were trypsinized and re-plated for functional assays 24 h later. For comparing different transposon sizes, cells were transfected with 1.0 μg of transposase and transposon plasmids mAppleT2APuro (0.65 μg) or mApple-β-Geo (1.1 μg) or mAppleT2APuro pAdeasy (1.8 μg) to keep the number of transposon plasmids equivalent between transfections. Additional DNA was added to a final amount of 2.8 μg using pUC19 to keep total DNA constant between transfections.

The plasmid-to-plasmid assay was performed essentially as described (Chatterji, M., et al. Mol Cell Biol 2006 26:1558-1568). HEK 293T cells were plated at a density of 500,000 cells per well in a six-well plate and transfected the next day with 0.5 μg transposase plasmid, 1 μg transposon plasmid, and 1.5 μg target plasmid DNA using Lipofectamine 3000 (Invitrogen) according to the manufacturer's instructions. Cells were collected 48 h later, and LMW DNA isolated using a modification of Birnboim and Doly, 1979 (Birnboim, H. C., et al. Nucleic Acids Res 1979 7:1513-1523). After the third ethanol-precipitation step, the pellet was redissolved in 25 μl of TE buffer (0.01 M Tris-HCl pH 8.0, 1 mM sodium EDTA) and used for electroporation of ElectroMAX DH10B cells (Invitrogen). Cells were subsequently plated on both KTS (30 μg/ml kanamycin, 12 μg/ml tetracycline, 30 μg/ml streptomycin) plates (0.4-0.8 ml) and 30 μg/ml Kan plates (1×104 dilution); after 24 h, colonies were counted, and activity quantified by dividing the number of KTS colonies by the number of Kan colonies.

### Excision Assay

Excision and rejoining (excision) assay analysis was performed as described by previously (Chen, Q., et al. Nat Commun, 2020 11:3446; Wilson, M. H., et al. Molecular Therapy, 2007 15:139-145). Plasmid DNA was recovered from transfected cells 24 h after transfection and subjected to excision PCR analysis (primers listed in supplementary table). PCR products were visualized using agarose gel electrophoresis and ethidium bromide staining. Excision bands were excised, and transposition was confirmed via DNA sequencing as described previously (Chen, Q., et al. Nat Commun, 2020 11:3446; Wilson, M. H., et al. Molecular Therapy, 2007 15:139-145).

### Colony Count Assay

One day after transfection, 2500 cells were replated on 10-cm dishes in growth media plus G418 (700 μg/ml) or puromycin (3 μg/ml) and selected for 10 days. Cell colonies were then fixed, stained with methylene blue and counted as described previously (Luo, W., et al. Nucleic Acids Res 2017 45:8411-8422).

For piggyBat: cell culture, transfection, and colony count assays were carried out. HEK293T cells were cultured using standard procedures. For transfection, cells were seeded at a density of 0.5M cells per well in a six-well plate and transfected with 10 ng of transposon (donor) and 20 ng of transposase (helper) plasmid DNA using Lipofectamine 3000 (Invitrogen), according to manufacturer's instructions. 48 hrs post-transfection cells were trypsinized and diluted in 100 mm dishes followed by selection with 2 ug/ml of puromycin for 11 days with media changes every 3 days. Plates were then fixed using 4% formaldehyde in phosphate-buffered saline (PBS), stained with 1% methylene blue in PBS and counted.

Quantitation of Transposon Copy Number with Droplet Digital PCR

Droplet digital PCR was used for transposon and RNAse P copy number. HT-1080 cells were transfected and selected as described above. After a minimum of two weeks of selection, genomic DNA was isolated as above. To reduce episomal transposon DNA, isolated DNA was treated with restriction enzyme Dpn I for which mammalian genomic DNA cleavage is blocked by overlapping CpG methylation. Ten ng of genomic DNA was used to amplify the neomycin resistance or RNase Ps genes (all primers, supplementary table). Primer/probe concentration was 900 nM/250 nM. Neo primers/probe and RNase P primers/probe were placed in one tube with channel 1 for Neo-FAM and channel 2 for RNAse P-Hex to reduce pipetting errors. The Neo copy number per RNAse P was directly calculated by Neo copy number divided by RNAse P copy number in 20 μl reaction.

### Genome-Wide Sequencing Library Preparation

HCT116 cells were transfected with transposase plasmids pCMV-PB, hyPB, Δ74PB-2CD, Δ74hyPB-2CD and transposon plasmids PB-SRT-Puro LE-RE, PB-SRT-Puro LE-LE using lipofectamine LTX in 100 mm dishes. Next day after transfection, cells were split into four 100 mm dishes containing 3 μg/ml of puromycin medium. After two weeks selection with puromycin, cells were collected, and RNA was prepared using an RNeasy RNA mini kit (Qiagen). Four micrograms of total RNA were used were prepared cDNAs using M-MLV reverse transcriptase, RNase H minus, point mutant which is used in cDNA synthesis with long RNA templates of more than 5 kb (Promega #M3681) and primer SMART-dT18VN. cDNAs were PCR amplified with four primers located in transposon areas, SRT-PAC-F1, SRT-Seq P1, SRT-Seq P2, and SRT-Seq P3 and one primer located in Smart-dT18VN, being Smart. Amplified cDNAs were purified using PCR/Gel purification column (Macherey-Nagel #740609). 500 ng of PCR amplicons were fragmented and tagged with Illumina DNA Prep (Illumina #20060060). Tagged DNA fragments were further PCR amplified using Read1-TnME, and Read2-R-5′TIR and PCR amplicons were 100-500 bp size-selected with 2% agarose gel. Dual index primers (NEB #E7780S) for sequencing on Illumina MiSeq and NovaSeq platforms were added by amplifying the tagmented samples using Q5 polymerase (NEB #M0544S) following NEBnext protocol (NEB #E7645S—Section 4.1). The PCR products were purified using AMPure XP beads (Beckman Coulter #A63881) using 0.9× bead volume. The quality of the final library was verified on TapeStation.

### Trimming of the Raw Reads.

Raw reads from each sample were trimmed twice using cutadapt (Martin, M. EMBnet J. 2011, 17:3). First trimming was to remove the sequencing adapters and retain the Tn5 tagmentation sequence and transposon inverted repeat (TIR) sequence for analysis of the transposon integration features. Second trimming removed the constant TIR sequences to enable efficient alignment of the reads to the genome. Specific trimming parameters for PB are in the supplementary version of computational methods (PDF of all code) or online in the associated github page (https://github.com/HaaseLab/piggyBac_mutants).

### Sequence Logo of Integration Preferences.

The fastq file of the second read (R2) containing TIR sequence after removal of sequencing adapters was loaded into R and analyzed using the ShortRead package (Morgan, M., et al. Bioinformatics 2009 252607-2608). The length of each read was trimmed to same length and reads containing perfectly matching TIR sequence at their five prime ends were selected. Nucleotide frequency per position was calculated and plotted using ggplot and ggseqlogo.

### Peak Calling

The TIR sequence was removed and reads were aligned to the human genome (Gencode GRCh38.p5.v24) in paired-end mode using Hisat aligner (Kim, D., et al. Nat Biotechnol 2019 37:907-915) with standard settings. The bam files were loaded into R using custom function implementing Rsamtools, BSgenome.Hsapiens.UCSC, GenomicRanges, GenomicAlignments, and data.table. Briefly, perfectly mapping and perfectly paired primary alignment reads were loaded into R, and each read pair converted into a single read fragment. Only unique genomic read fragments smaller than the median library size were kept for further analysis. This data was used to calculate stranded coverage that was consecutively used to find peaks using slice( ) function from GenomicRanges package. Any genomic position with minimum of five unique fragments coverage was identified to be a peak and used in consecutive analyses. Annotated peak list for each sample is provided with GEO (GSE201914).

### Results

piggyBac transposase is multiply phosphorylated in mammalian cells. One surprising aspect of the first piggy Bac transpososome structures was the absence of structure for the first 116 N-terminal residues, representing 20% of the 594 amino acid transposase, indicating that this region was disordered in both the transposon end and strand-transfer complexes (Chen, Q., et al. Nat Commun, 2020 11:3446). Assignment of function to this segment remained elusive as observed protein/DNA interactions in the structures accounted for the available footprinting data (Morellet, N., et al. Nucleic Acids Res, 2018 46:2660-2677) and the estimated pI of 4.3 for this protein segment suggested that it was unlikely to be involved in binding to DNA. A low pI for the N-terminal region of the transposase is a common property of members of the large superfamily of piggyBac-like elements although these regions cannot be aligned (Bouallegue, M., et al. Genome Biol Evol 2017 9:323-339). However, many piggyBac-like elements possess multiple casein kinase II (CKII) phosphorylation motifs, S/T-D/E-X-E/D (Ubersax, J. A., et al. Nat Rev Mol Cell Biol 2007 8:530-541), in this region. In particular, both piggy Bac and piggy Bat, two transposons with demonstrated activity in mammalian cells (Mitra, R., et al. Proc Natl Acad Sci USA 2013 110:234-239), have several CKII consensus sites within their transposase N-termini (FIG. 1B).

Mass spectrometry was used to evaluate the phosphorylation status of the piggy Bac transposase expressed and purified from HEK293F cells. The absence of basic residues in the N-terminus prevented the use of trypsin digestion, but digestion of purified piggy Bac transposase (PB1-539) with Asp-N protease yielded phosphorylated peptides that could be confidently identified by LC-MS/MS. Furthermore, the detection of corresponding pairs of phosphorylated vs. non-phosphorylated peptides allowed estimation of the extent of phosphorylation. The data indicated that Ser17 and Ser35 were fully phosphorylated (FIG. 1B), whereas Ser41 was phosphorylated in ˜20% of spanning peptides; in this same region, Ser26 lacked detectable phosphorylation.

Transposase N-terminal phosphorylation inhibits transposition in cells. To determine if N-terminal phosphorylation affected the activity of the piggy Bac transposase in human cells, we used a donor plasmid-to-target plasmid transposition assay (FIG. 1C) (Chatterji, M., et al. Mol Cell Biol 2006 26:1558-1568; Zhang, Y., et al. Nature 2019 569:79-84). We compared WT piggy Bac transposase to mutants with the three serine residues mutated to Ala (“AllStoA”) or individually to Pro (S17P, S35P, or S41P). A mutant in which the three Ser residues were changed to Glu (“AllStoE”) was also tested as possible mimics of phosphorylation. As shown in FIG. 1D, all mutations increased transposition activity relative to WT, with the highest activity (4-fold increase) observed with the AllStoA mutant. Although all three individual Ser-to-Pro mutations contributed to the activity increase, the most important contributor appeared to be S35. The AllStoA piggy Bac transposase also exhibited increased activity as assessed in a colony count assay that measured integration of a neomycin resistant transposon (FIG. 1E) (Wilson, M. H., et al. Molecular Therapy, 2007 15:139-145).

Predicted structure of full-length piggy Bac transposase suggests that N-terminus phosphorylation inhibits DNA binding. To understand how the N-terminal region of the piggy Bac transposase might be affecting its activity, we took advantage of the predictive power of AlphaFold-Multimer (Evans, R., et al. bioRxiv, 2022). Models were generated for the dimeric full-length piggy Bac transposase that were ranked by pLDDT score (local distance difference test) and, in all models, segments of the first 116 amino acids folded with defined secondary structures that packed against the rest of the protein (FIG. 2A,2B). Common to all models was an α-helix from Asp7-Leu15 that pointed towards the transposon end binding site indicated in the cryo-EM structures. Also predicted was an α-helix (Ser76-Arg81) that, together with a short b-hairpin (b-strands Thr89-Gly92 and Lys95-Trp98), extended a β-stranded insertion domain in the transposase catalytic core suggesting that they might participate in target DNA binding by increasing the target binding protein/DNA surface observed in the cryo-EM structure.

Of particular interest, several of the highest confidence models predicted that a stretch of the first 116 residues is arranged in trans, i.e., residues from one transposase monomer cross over and pack against the second monomer. In such an arrangement (FIG. 2A), all three N-terminal Ser residues identified as being phosphorylated were positioned such that they would conflict with DNA binding: S17 was positioned where bp 8 of the transposon end is located, and S35 and S41 were within the target binding site. This suggested the possibility that negatively charged phosphorylated N-terminal Ser residues may act as competitors to DNA binding, an effect that would be diminished if phosphorylation were prevented or by N-terminal truncation. We also noted that, in these models, three of the seven mutations that are responsible for “hyperactive piggyBac”, hyPB (Wachtl, G., et al. Int. J. Mol. Sci., 202223:10317; Jin, Y., et al. Proc Natl Acad Sci USA, 2017 114:7408-7413; Yusa, K., et al. Proc. Natl. Acad. Sci. U.S.A., 2011 108:1531-1536), lie within or close to the predicted interaction surfaces between the N-terminal domain and the rest of the transposase (I30V, M282V, S103P, FIG. 2B).

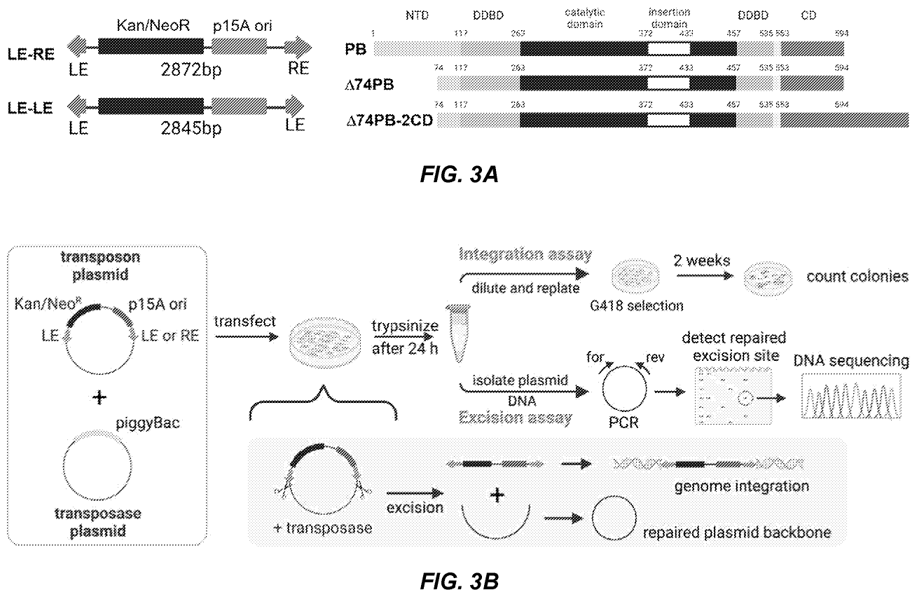

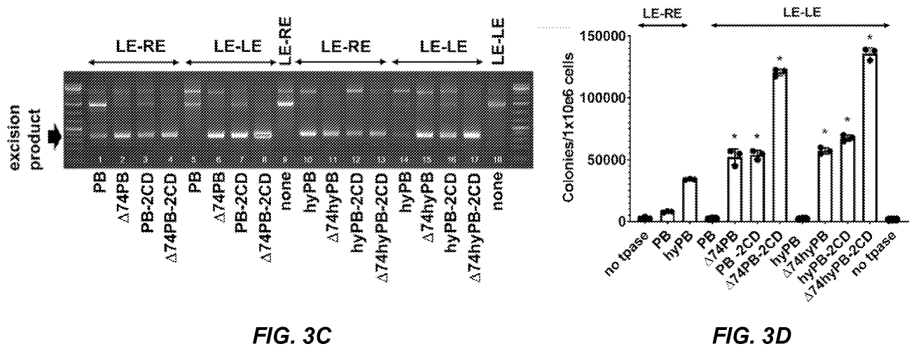

Deletion of the N-terminal 74 residues of the piggy Bac transposase overcomes inhibition of transposition. To evaluate the effect on transposition activity of preventing phosphorylation, two N-terminally truncated mutants of the piggy Bac transposase, Δ74PB and Δ104PB, were generated and compared to the WT transposase (PB). To test the effect of the shorter N-terminal truncation, Δ74PB transposase was tested for transposon excision and integration in the left end-right end (LE-RE) and left end-left end (LE-LE) format (FIG. 3A). Excision was evaluated using a PCR-based excision assay, and colony count assays served as proxy for integration of a neomycin-resistant (NeoR) transposon (FIG. 3B) (Wilson, M. H., et al. Molecular Therapy, 2007 15:139-145). Using symmetrical LE-LE transposon ends, WT PB exhibited little to no excision (FIG. 3C, lane 5), and hyPB showed very low excision activity (FIG. 3C, lane 14); neither was capable of LE-LE integration (FIG. 3D). However, Δ74PB and Δ74hyPB are not only capable of LE-LE excision (FIG. 3C, lanes 6 and 15), they both exhibit higher integration activity than PB or hyPB transposases with LE-RE transposons (FIG. 3D).

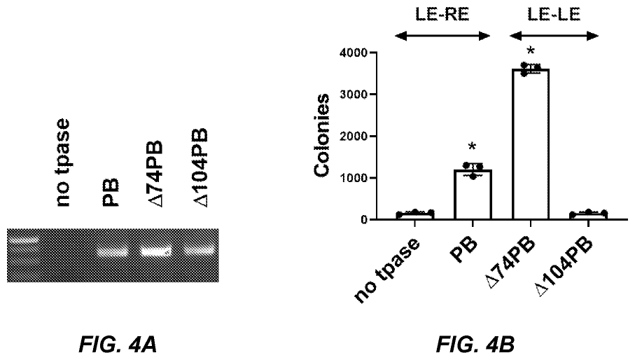

Deletion of the N-terminal 104 residues of the piggy Bac transposase results in a phenotype of excision active/integration inactive activity on LE-LE transposon DNA. A particular advantage of using piggyBac for genome engineering is its seamless excision as the excision site can be repaired by the host without leaving permanent changes called excision footprints. Previous mutation of the piggy Bac transposase resulted in an excision active/integration inactive (exc+/int−) transposase that can excise LE-RE transposons but cannot reintegrate them (Li, X., et al. Proc Natl Acad Sci USA, 2013 110:E2279-2287). It was considered that the Alphafold2 structural prediction that residues 75-100 may participate in target binding by extending the target binding protein/DNA surface (FIG. 2A,2B). Therefore, the N-terminal region beyond 474 was further truncated to generate Δ104PB and its LE-LE excision and integration activity was evaluated. In contrast to Δ74PB and satisfyingly consistent with AlphaFold2 predictions, Δ104PB was capable of excising but not integrating LE-LE transposon DNA into the human genome (FIG. 4A,4B). Repair of the donor plasmid was unaffected as sequencing of the excision PCR product demonstrated precise reconstitution of the TTAA upon excision. Therefore, Δ104PB represents an exc+/int− transposase for LE-LE transposons.

The N-terminus is required for transposase dimerization prior to transposon end binding. Although an attempt was made to explore the role of the piggyBac transposase N-terminus using purified proteins, no soluble N-terminally truncated transposases was recovered under conditions successfully used for the WT transposase in EXPI293F cells (Chen, Q., et al. Nat Commun, 2020 11:3446). To circumvent this, a TEV protease cleavage site was introduced between resides 74 and 75 which allowed expression and purification of a full-length protein and then proteolytical removal of the first 74 amino acids. Evaluation of the oligomerization state by analytical ultracentrifugation revealed that, like WT, PB1-558 transposase was a monodisperse dimer with a species at a sedimentation coefficient of 6.72 S corresponding to 125 kDa (93% of the absorbance signal; in blue, FIG. 2C). Conversely, the PB74-539 transposase generated by proteolytic cleavage was a monomer with a dominant species at a sedimentation coefficient of 3.58 S corresponding to 53.4 kDa (88% of the absorbance signal; FIG. 2C). This result supported the in trans models for piggy Bac transposase dimerization and is consistent with observations that the full-length transposase is a dimer even prior to transposon end binding and synapse (Chen, Q., et al. Nat Commun, 2020 11:3446; Jin, Y., et al. Proc Natl Acad Sci USA, 2017 114:7408-7413). The trans binding segment of the N-terminus is displaced upon transposon end binding and after synapse formation the extensive network of protein-DNA interactions seen in the cryo-EM structures now hold the dimer together. This would also explain why the N-terminal region was structurally disordered in the cryo-EM maps.

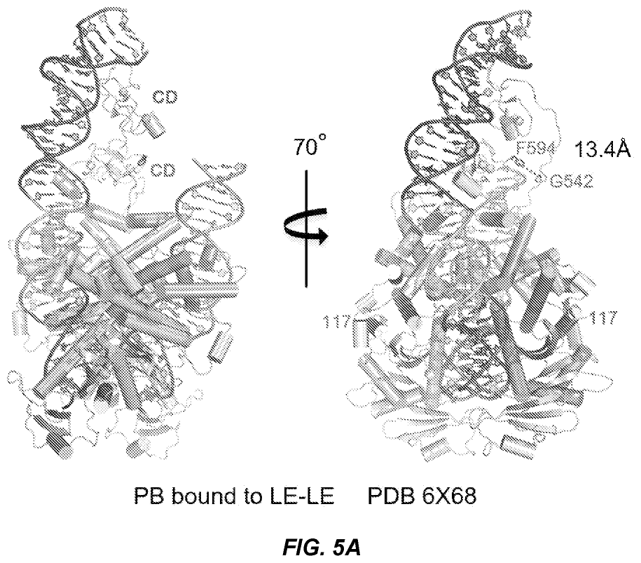

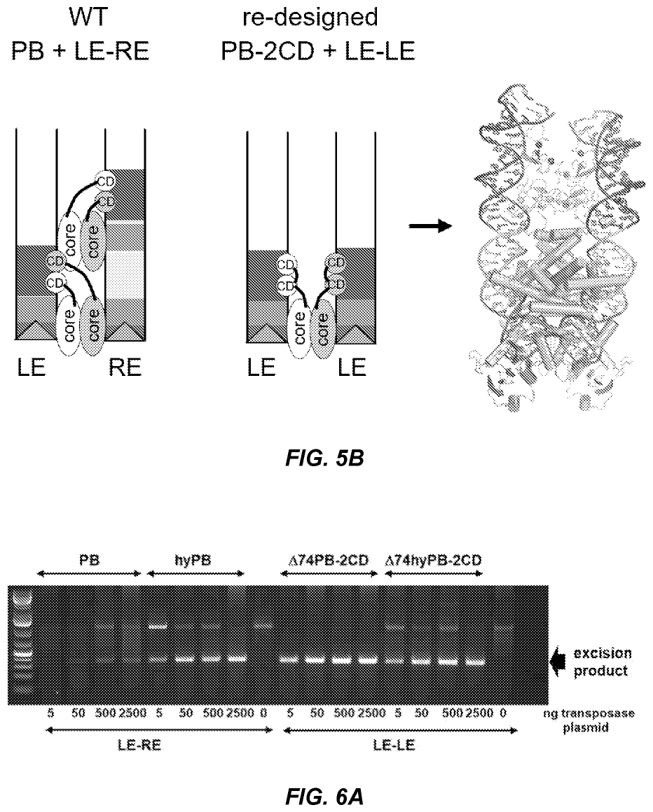

Addition of a second C-terminal domain to the piggy Bac transposase overcomes the lack of activity on symmetric transposon ends. The cryo-EM structures of the dimeric piggyBac transposase bound to two oligonucleotides representing two transposon LEs showed that the multidomain enzyme recognizes motifs within its ends in a modular fashion (Chen, Q., et al. Nat Commun, 2020 11:3446). The tip of each transposon end is bound by the central catalytic domain (residues 264-456), and a DNA motif just inside the transposon ends (FIG. 1A) is recognized by a bipartite DNA binding domain (residues 117-263 and 457-535). However, the strongest binding affinity is conferred by the cysteine-rich C-terminal domain (“CD”; residues 553-594) that dimerizes as it binds to a 19-bp palindrome. Although both transposon ends have these palindromes, the fact that the dimeric transposase can supply only two CDs resulted in a surprisingly asymmetric structure (FIG. 5A) with the palindrome of one LE bound by two CDs while the palindrome of the other LE was unbound and disordered in the electrostatic potential density map. This result suggested that binding of the palindrome requires dimerization of the CD. As the palindromes are asymmetrically arranged on the LE and the RE (FIG. 1A), it was previously proposed that the most straightforward model of a transpososome assembled on the active LE-RE ends is a tetramer (FIG. 5B) as it is the minimal oligomerization state that can provide two CDs to each of the two palindromes (Chen, Q., et al. Nat Commun, 2020 11:3446). A need for the binding affinity or arrangement provided by four CDs and two palindromic sequences would explain why a modified piggy Bac transposon with symmetrical LE-LE ends cannot be excised or integrated in cells as it allows only for the binding of a dimer and hence the inactive transpososome contains only two CDs (Chen, Q., et al. Nat Commun, 2020 11:3446).

Considering the in-cell requirement for asymmetric ends, it was hypothesized that transposition using symmetrical LE-LE transposon ends in cells could be accomplished by appending a second CD, representing residues 543-594 immediately following the terminal residue of the WT transposase, F594 (FIG. 5B). Such a modified transposase should be able to supply two CDs to the 19-bp palindrome on a single LE (FIG. 1A). The piggyBac transpososome structures indicated that the addition of a second CD could be accommodated since a long, partially disordered linker connects residues 535 and 553 of the CD, and there are no protein-protein interactions between either of the CDs and the rest of the transposase (FIG. 5A). When this new transposase was tested in human cells, the effect of second C-terminal domain addition on excision and integration by piggyBac (PB-2CD) and hyperactive piggyBac (hyPB-2CD) was very similar to that observed for the Δ74 truncation: both modified transposases were active on symmetrized LE-LE transposons (FIG. 3c, lanes 7 and 16; FIG. 3d), a property not exhibited by the WT piggy Bac transposase (PB) or hyPB (FIG. 3C, lanes 7 vs. 5 and 16 vs. 14; FIG. 3D).

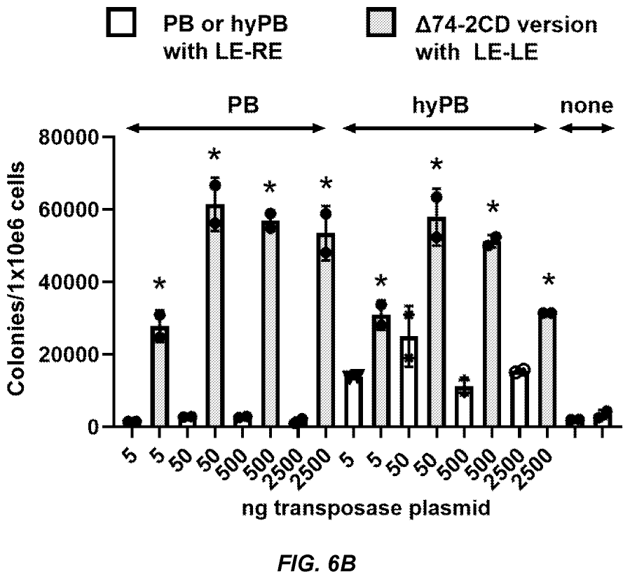

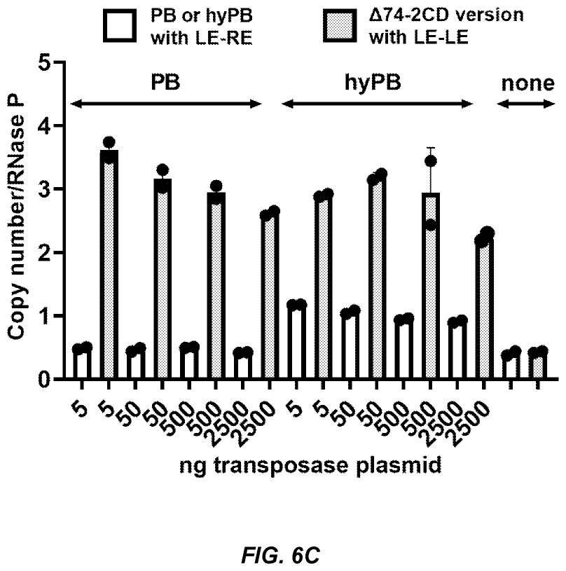

Combining the Δ74 and 2CD modifications leads to a highly active transposase on LE-LE transposons. When the two transposase modifications, Δ74 and the 2CD addition were we combined into one transposase, both Δ74PB-2CD and Δ74hyPB-2CD exhibited high excision activity (FIG. 3C, lanes 8 and 17) and additive integration activity when compared to Δ74- or -2CD transposases alone with LE-LE transposons (FIG. 3D). Excision and integration activities of the Δ74PB-2CD and Δ74hyPB-2CD transposases over a range of transposase doses (5 ng to 2.5 μg of plasmid encoding the transposase) was also evaluated in combination with a fixed amount of transposon DNA (1.5 μg) (FIG. 6A,6B). At 500 ng of transposase plasmid, there was a dramatic 20-fold increase in integration activity by Δ74PB-2CD with LE-LE relative to the WT piggyBac transposase (PB) with LE-RE (FIG. 6B) and a 5-fold increase in integration activity for over that of the benchmark combination of hyPB with LE-RE (FIG. 6B).

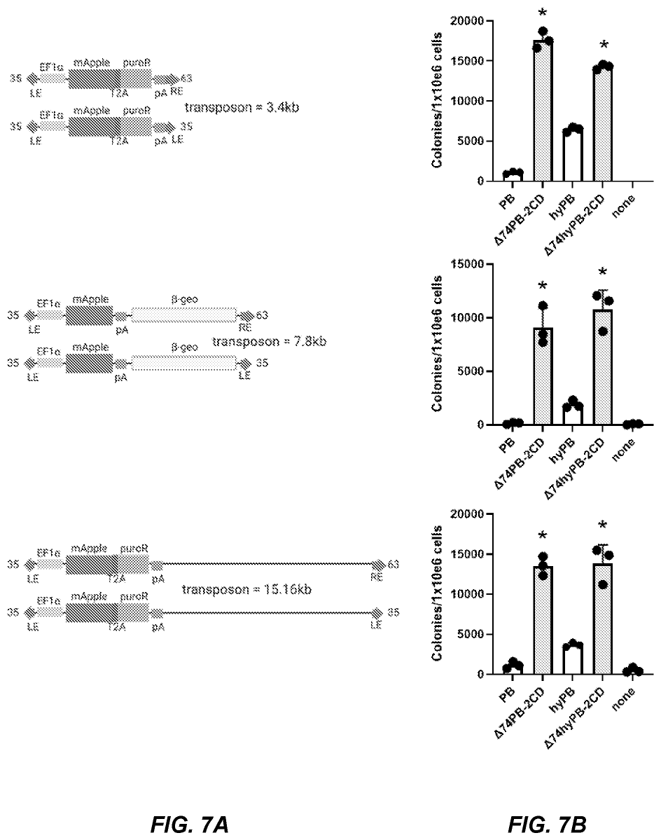

To further characterize the effect of combining the two transposase modifications, it was confirmed that Δ74PB-2CD with LE-LE transposon ends reconstituted the TTAA target site after excision by sequencing the excision PCR product, thus validating the key molecular signature of piggyBac's seamless excision. The copy number of integrated transposons was also determined in human cells when using 1 μg of transposase plasmid with 1.5 μg of transposon DNA. As expected and consistent with excision and integration analysis, there were more integration events for the Δ74PB-2CD and Δ74hyPB-2CD transposases with LE-LE transposons than with the WT piggyBac transposase (PB) or hyPB with LE-RE transposons (FIG. 6C). Δ74PB-2CD or 474hyPB-2CD also outperformed their counterparts in integrating a range of small to very large transposons (3.4 kb, 7.8 kb, and 15.1 kb) in human cells (FIG. 7), indicating that these modifications overcome the negative regulation mediated by N-terminal phosphorylation and asymmetric transposon ends.

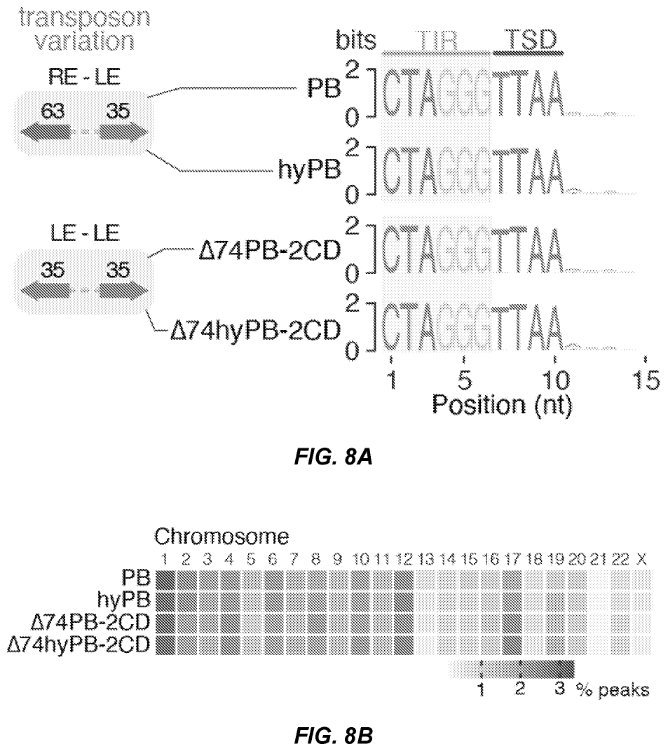

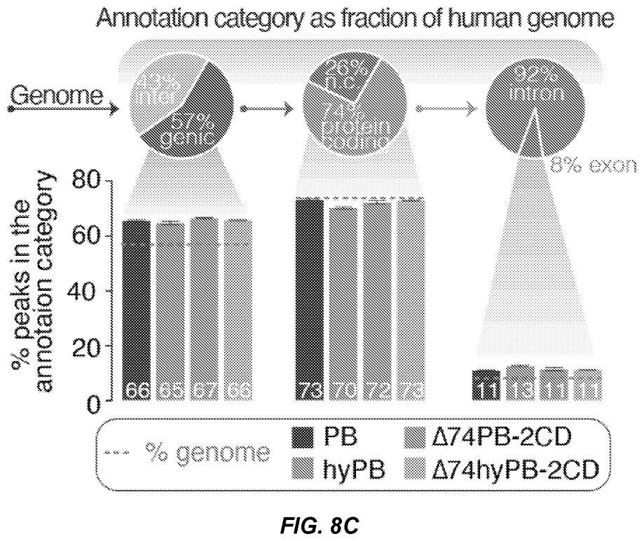

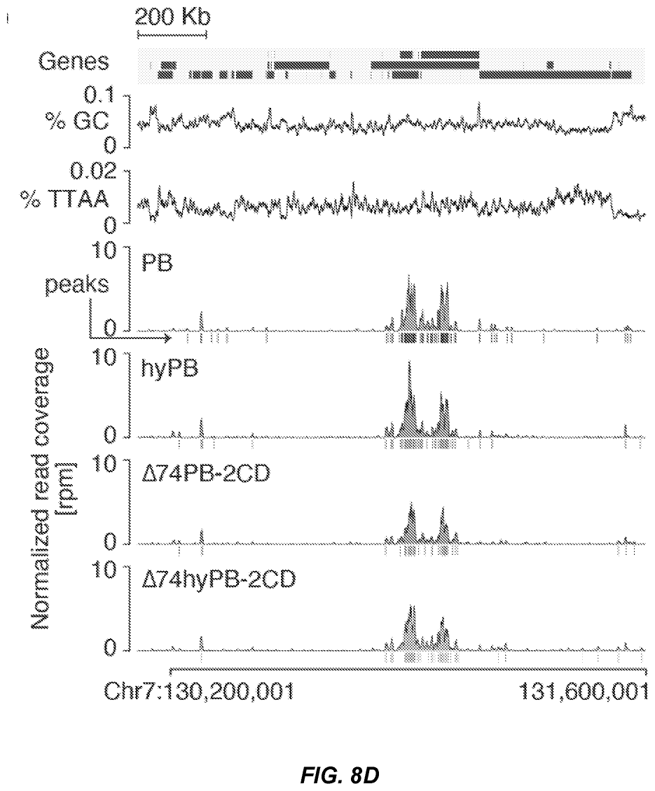

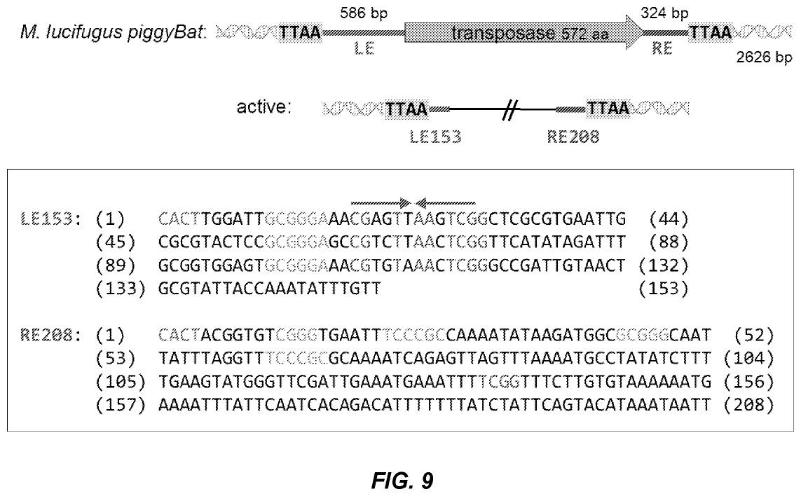

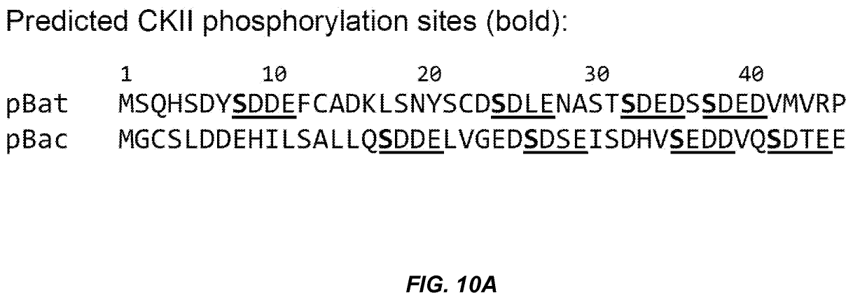

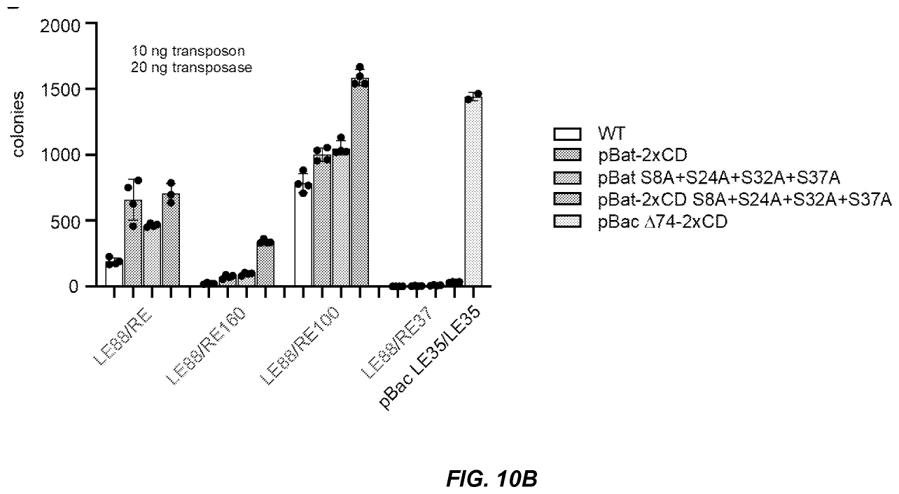

Δ74PB-2CD transposase demonstrates an unaltered integration profile in human cells. It has been previously reported that modifications to the piggy Bac transposase can result in loss of integrity of precise excision and target site duplication (Helou, L., et al. J Mol Biol 2021 433:166805). To evaluate the integration site profile of the modified piggy Bac transpososomes (the combination of modified transposases with a symmetric LE-LE transposon), self-reporting transposon technology was used to probe potential alterations in integration site profiles in HCT116 cells (Moudgil, A., et al. Cell 2020 182:992-1008 e1021). There was precise and consistent excision and target site duplication for all transpososomes tested (FIG. 8A). Genome-wide analyses of integration sites revealed a broad and consistent distribution across all chromosomes for WT and the modified transpososomes (FIG. 8B). Further annotation by genomic features showed comparable preferences for different genomic regions for all piggy Bac transpososomes (FIG. 8C). To characterize individual insertion sites in more detail, the genome was split into one mega-base regions and plotted the normalized abundance of insertion sites for WT and the modified transposases. A candidate genomic region revealed similar insertion patterns (FIG. 8D). Overall, genome-wide analysis of insertion sites in human cells revealed that all redesigned piggyBac transposases maintained precise excision and target site duplications when used in combination with symmetric LE-LE transposons.

In cell based transposition assays, modifications of both the piggy Bat transposase that involve the mutation of four N-terminal residues S8 to A, S24 to A, S32 to A and S37 A and the addition of a second C-terminal domain CD resulting in the protein sequence SEQ ID NO:33.

The growing list of high-resolution three-dimensional structures of DNA transposases in complex with the specific DNA upon which they act has provided unprecedented insight into their mechanisms of action (Hickman, A. B., et al. Chemical Reviews 2016 116:12758-12784; Liu, C., et al. Nature 2019575:540-544; Kaczmarska, Z., et al. Mol Cell. 2022 82 (14): 2618-2632.e7; Ghanim, G. E., et al. Nat Struct Mol Biol. 2019 26 (11): 1013-1022). For the piggy Bac transposase, a particularly revealing observation was that the way the transposase binds the tip of the transposon hairpin intermediate structurally mimics how the target site is bound, establishing an unanticipated link between its mechanisms of excision and integration (Chen, Q., et al. Nat Commun, 2020 11:3446).

Here, more insight was attained into piggy Bac transposition in mammalian cells using the AlphaFold2 system (Evans, R., et al. bioRxiv, 2022; Jumper, J., et al. Nature 2021 596:583-589) in combination with results from cellular and biochemical assays. Of particular interest was the role of the N-terminal 116 residues which were disordered in the cryo-EM structures (Meir, Y. J., et al. FASEB journal 2013 27:4429-4443). CKII phosphorylation sites were identified on specific serine residues within the N-terminal region of the transposase and verified that mutation of these residues to prevent phosphorylation stimulates transposition in mammalian cells. The AlphaFold-generated models suggested that CKII phosphorylation may interfere with the binding of transposon ends and target DNA, thus mechanistically explaining the inhibitory effect.

Transposase phosphorylation has been reported previously; for example, Protein Kinase A dependent inhibition of Mos1 has been observed (Bouchet, N., et al. Nucleic Acids Res 2014 42:1117-1128), although only a small fraction of the Mos1 transposase appears phosphorylated in S2 insect cells. The ubiquitous presence of CKII phosphorylation motifs within the sequences of piggyBac-like element transposases and the demonstrated phosphorylation of the piggy Bac transposase at multiple sites suggests that CKII most likely plays a similar role in downregulating these mobile genetic elements within their native host species. CKII is a constitutive kinase, primarily but not exclusively located in the nucleus (Venerando, A., et al. Biochemical Journal 2014 460:141-156). These results suggest that transposon downregulation can now be added to the extraordinary pleiotropy of CKII. Therefore, phosphorylation can be used as a way to regulate piggy Bac activity or the timing of transposition in mammalian cells, for example by changing the CKII motifs to those of other kinases of interest.

The AlphaFold2 predictions also provided a framework for understanding the observation that Δ104PB is an exc+/int− transposase on LE-LE transposon DNA. This deletion mutant of the piggyBac transposase may be particularly valuable when 474PB-2CD or Δ74hyPB-2CD are used for high efficiency transposon integration, as those LE-LE transposons can still be excised by providing Δ104PB if needed in downstream applications. This therefore complements previous mutations of the piggy Bac transposase that resulted in exc+/int− activity on LE-RE transposons (Li, X., et al. Proc Natl Acad Sci USA, 2013 110: E2279-2287). We conclude that the combination of AlphaFold2 with experimental structures can be particularly powerful by providing hypotheses as to the function of parts of molecular assemblies that are invisible in experimental structures due to disorder.

An example of the power of this synergistic relationship between experiment and prediction was a question left unanswered by our cryo-EM structures. Biophysical data indicated that the piggy Bac transposase is a dimer when expressed in mammalian cells (Jin, Y., et al. Proc Natl Acad Sci USA, 2017 114:7408-7413), even before the formation of synaptic complex. Yet the almost entirely polar protein-protein interface between the two monomers is quite small at 644 A2 and seemed insufficient to explain dimerization prior to DNA binding. AlphaFold2 prediction suggested that the N-terminus might provide critical monomer-monomer interactions prior to DNA binding, and this was confirmed using purified proteins and sedimentation analysis. It was suspected that, upon synaptic complex formation, the bound transposon ends may displace the N-terminus while maintaining transposase dimerization.

The native piggyBac transpososome can be rationally simplified to a presumed symmetric dimer via the addition of a second CD to the transposase to allow transposition using symmetric LE-LE transposon ends upon which the WT transposase is inactive. The surprisingly robust activity was further increased by the Δ474 truncation. This redesign of the piggyBac transpososome to include Δ74PB-2CD in combination with symmetric LE-LE ends has produced a novel highly active transpososome with apparently unaltered excision and integration fidelity. Relative to the WT piggy Bac transposase and hyPB, the redesigned piggy Bac transpososomes exhibit unaltered integration site profiles with no loss of integrity of transposon ends. The Δ104 version also offers the ability to excise and not re-integrate symmetric end transposon DNA if needed. Such exc+/int− transposases have been used previously to created transgene-free iPSCs and for selection of gene-modified cells wherein the selection cassette can subsequently be removed leaving the genome intact (Kaji, K., et al. Nature 2009 458:771-775; Yusa, K., et al. Nature 2011 478:391-394). Ultimately, these simplified piggyBac transpososomes provide novel scaffolds that may allow for further engineering to achieve other outcomes such as high efficiency targeted transposon integration in mammalian cells, a goal which thus far remains elusive. In particular, reducing the number of appended targeting domains from four in a homotetrameric piggy Bac transpososome to only two in a presumed dimeric Δ74PB-2CD transpososome may be a particularly attractive benefit.

The results here indicated that the piggy Bac transposition system as natively isolated from the cabbage looper moth has layers of mechanistic complexities that seem likely to serve to downregulate its activity in a host. When assessed in mammalian cells, neither the fidelity nor the level of activity carried out by its transposase is fundamentally perturbed by the removal of the large part of the N-terminal region or by symmetrizing the transposon ends (as long as binding affinity is compensated for).

One aspect that unites otherwise surprisingly diverse transpososome architectures is their modularity. It appears that the RNAseH-like core can accommodate a variety of different insertions of other domains and the fusion of variable numbers of different DNA binding domains (often Zn finger variants). These architectures, if characterized precisely by three-dimensional structures, could provide insight for other transpososomes. Modularity offers rich re-engineering possibilities. Furthermore, the rapid development of computational tools such as Rosetta allows for the design of new protein/protein interfaces to create stable assemblies with novel functions (Kuhlman, B. J. Biol. Chem. 2019 294:19436-19443). One envisions a future in which the combination of experimental and computational tools will be used to generate novel DNA transpososomes to add to the genomic tool kit available for genomic and clinical applications.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A modified transposase, comprising a core piggyBac transposase lacking one or more of the N-terminal 74 amino acids, having a duplication of the C-terminal 53 amino acids, or a combination thereof.

2. The modified transposase of claim 1, wherein the core piggyBac transposase comprises the amino acid sequence SEQ ID NO:1.

3. The modified transposase of claim 2, wherein the piggyBac transposase comprises the formula:
Xₐ-SEQ ID NO:2, 1.
wherein Xₐ is 0 to 70 aa of the amino acid sequence SEQ ID NO:3.

4. The modified transposase of claim 3, wherein the piggyBac transposase comprises the amino acid sequence SEQ ID NO:4, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:4.

5. The modified transposase of claim 2, wherein the piggyBac transposase comprises the formula:
SEQ ID NO:1-X₀-X_{b}, 2.
wherein X_{b} is 40-53 aa of the amino acid sequence SEQ ID NO:5, and wherein X₀ is a linker comprising 0-20 amino acid residues.

6. The modified transposase of claim 2, wherein the piggyBac transposase comprises the formula:
Xₐ-SEQ ID NO:2-X₀-X_{b}, 3.
wherein Xₐ is 0 to 70 aa of the amino acid sequence SEQ ID NO:3, wherein X_{b} is 40-53 aa of the amino acid sequence SEQ ID NO:5, and wherein X₀ is a linker comprising 0-20 amino acid residues.

7. The modified transposase of claim 6, wherein the piggyBac transposase comprises the amino acid sequence SEQ ID NO:6, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:8.

8. The modified transposase of claim 1, wherein the core piggy Bac transposase comprises the amino acid sequence SEQ ID NO:7, wherein X₁ is G or C, wherein X₂ is S or N, wherein X₃ is S or P, wherein X₄ is I or V, wherein X₅ is Q or R, wherein X₆ is T or A, wherein X₇ is Q or R, wherein X₈ is T or A, wherein X₉ is S or R, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

9. The modified transposase of claim 8, wherein the piggy Bac transposase comprises the amino acid sequence SEQ ID NO: 11, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

10. The modified transposase of claim 8, wherein the piggyBac transposase comprises the amino acid sequence SEQ ID NO: 12, wherein X₁ is G or C, wherein X₂ is S or N, wherein X₃ is S or P, wherein X₄ is I or V, wherein X₅ is Q or R, wherein X₆ is T or A, wherein X₇ is Q or R, wherein X₈ is T or A, wherein X₉ is S or R, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

11. The modified transposase of claim 8, wherein the piggyBac transposase comprises the amino acid sequence SEQ ID NO: 13, wherein X₁₀ is I or V, wherein X₁₁ is I or V, wherein X₁₂ is S, P, or T, wherein X₁₃ is N or S, wherein X₁₄ is G or S, wherein X₁₅ is M or L, wherein X₁₆ is M or V, wherein X₁₇ is S or N, wherein X₁₈ is R or G, wherein X₁₉ is M or V, wherein X₂₀ is G or E, wherein X₂₁ is P or L, wherein X₂₂ is I or V, wherein X₂₃ is Q or L, wherein X₂₄ is K or N, wherein X₂₅ is N or D, wherein X₂₆ is S or G, wherein X₂₇ is N or K, wherein X₂₈ is K or I, wherein X₂₉ is N or S, wherein X₃₀ is S or L, wherein X₃₁ is K or R, wherein X₃₂ is Q, P, or R, and wherein X₃₃ is F or L.

12. The modified transposase of claim 1, wherein the piggyBac transposase has a deletion of 75 or more of the N-terminal 104 amino acids.

13. The modified transposase of claim 12, wherein the piggy Bac transposase comprises the amino acid sequence SEQ ID NO: 14, wherein X_{c} is 75 to 104 aa of the amino acid sequence SEQ ID NO: 15.

14. The modified transposase of claim 13, wherein the piggyBac transposase comprises the amino acid sequence SEQ ID NO:16, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 16.

15. The modified transposase of claim 12, wherein the piggy Bac transposase comprises the amino acid sequence SEQ ID NO: 17, wherein X_{c} is 75 to 104 aa of the amino acid sequence SEQ ID NO:15, wherein X_{b} is 40-53 aa of the amino acid sequence SEQ ID NO: 6, and wherein X₀ is a linker comprising 0-20 amino acid residues.

16. The modified transposase of claim 15, wherein the piggy Bac transposase comprises the amino acid sequence SEQ ID NO: 18, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:18.

17. The modified transposase of claim 1, wherein the core piggyBac transposase comprises the amino acid sequence SEQ ID NO:24.

18. The modified transposase of claim 1, wherein the piggy Bac transposase comprises the amino acid sequence SEQ ID NO:25, 26, or 27.

19. A modified transposase, comprising a core of piggy Bat transposase with the following point mutations: S8 to A, S24 to A, S32 to A, S37 to A and having a duplication of the C-terminal 81 amino acids,

20. The modified transposase of claim 19, wherein the core piggy Bat transposase comprises the amino acid sequence SEQ ID NO:33.

21. A system comprising:
(i) the modified transposase of claim 1, and
(ii) a modified piggyBat transposon that contains only the first 88 nucleotides of the transposon left end (LE) and only the first 100 nucleotides of the transposon right end (RE).

22. A modified piggyBat transposon that contains only the first 88 nucleotides of the transposon left end (LE) and only the first 100 nucleotides of the transposon right end (RE).

23. The modified piggyBat transposon of claim 22, wherein the LE does not comprise nucleotides 89 to 153 of SEQ ID NO:34, and wherein the RE does not comprise nucleotides 101 to 208 of SEQ ID NO:35.


SEQUENCE LISTING (37 sequences)

SEQ ID NO: 1 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 2 - protein, 520 aa, synthetic construct
SSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 3 - protein, 74 aa, synthetic construct
MGSSLDDEHILSALLQSDDELVGEDSDSEISDHVSEDDVQSDTEEAFIDEVHEVQPTSSGSEILDEQNVIEQPG

SEQ ID NO: 4 - protein, 521 aa, synthetic construct
MSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 5 - protein, 53 aa, synthetic construct
GTSDDSTEEPVMKKRTYCTYCPSKIRRKANASCKKCKKVICREHNIDMCQSCF

SEQ ID NO: 6 - protein, 574 aa, synthetic construct
MSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 7 - protein, 593 aa, synthetic construct
  VARIANT 1: note = Xaa is Gly or Cys
  VARIANT 3: note = Xaa is Ser or Asn
  VARIANT 25: note = Xaa is Ser or Pro
  VARIANT 29: note = Xaa is Ile or Val
  VARIANT 39: note = Xaa is Gln or Arg
  VARIANT 42: note = Xaa is Thr or Ala
  VARIANT 54: note = Xaa is Gln or Arg
  VARIANT 56: note = Xaa is Thr or Ala
  VARIANT 60: note = Xaa is Ser or Arg
  VARIANT 81: note = Xaa is Ile or Val
  VARIANT 89: note = Xaa is Ile or Val
  VARIANT 102: note = Xaa is Ser, Pro, or Thr
  VARIANT 112: note = Xaa is Asn or Ser
  VARIANT 164: note = Xaa is Gly or Ser
  VARIANT 184: note = Xaa is Met or Leu
  VARIANT 193: note = Xaa is Met or Val
  VARIANT 229: note = Xaa is Ser or Asn
  VARIANT 280: note = Xaa is Arg or Gly
  VARIANT 281: note = Xaa is Met or Val
  VARIANT 315: note = Xaa is Gly or Glu
  VARIANT 409: note = Xaa is Pro or Leu
  VARIANT 425: note = Xaa is Ile or Val
  VARIANT 496: note = Xaa is Gln or Leu
  VARIANT 500: note = Xaa is Lys or Asn
  VARIANT 504: note = Xaa is Asn or Asp
  VARIANT 508: note = Xaa is Ser or Gly
  VARIANT 537: note = Xaa is Asn or Lys
  VARIANT 564: note = Xaa is Lys or Ile
  VARIANT 570: note = Xaa is Asn or Ser
  VARIANT 572: note = Xaa is Ser or Leu
  VARIANT 574: note = Xaa is Lys or Arg
  VARIANT 590: note = Xaa is Gln, Pro, or Arg
  VARIANT 593: note = Xaa is Phe or Leu
XSXLDDEHILSALLQSDDELVGEDXDSEXSDHVSEDDVXSDXEEAFIDEVHEVXPXSSGXEILDEQNVIEQPGSSLASNRXLTLPQRTXRGKNKHCWSTSKXTRRSRVSALXIVRSQRGPTRMCRNIYDPLLCFKLFFTDEIISEIVKWTNAEISLKRRESMTXATFRDTNEDEIYAFFGILVXTAVRKDNHXSTDDLFDRSLSMVYVSVMSRDRFDFLIRCLRMDDKXIRPTLRENDVFTPVRKIWDLFIHQCIQNYTPGAHLTIDEQLLGFRGRCPFXXYIPNKPSKYGIKILMMCDSGTKYMINGMPYLGRXTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKXAKMVYLLSSCDEDASXNESTGKPQMVMYYNQTKGGVDTLDQMCSVMTCSRKTNRWPMALLYGMINIACINSFIIYSHNVSSKGEKVXSRKXFMRXLYMXLTSSFMRKRLEAPTLKRYLRDNISNILPXEVPGTSDDSTEEPVMKKRTYCTYCPSXIRRKAXAXCXKCKKVICREHNIDMCXSCX

SEQ ID NO: 8 - protein, 594 aa, unidentified
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 9 - protein, 594 aa, unidentified
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 10 - protein, 594 aa, unidentified
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 11 - protein, 521 aa, synthetic construct
  VARIANT 9: note = Xaa is Ile or Val
  VARIANT 17: note = Xaa is Ile or Val
  VARIANT 30: note = Xaa is Ser, Pro, or Thr
  VARIANT 40: note = Xaa is Asn or Ser
  VARIANT 92: note = Xaa is Gly or Ser
  VARIANT 112: note = Xaa is Met or Leu
  VARIANT 121: note = Xaa is Met or Val
  VARIANT 157: note = Xaa is Ser or Asn
  VARIANT 208: note = Xaa is Arg or Gly
  VARIANT 209: note = Xaa is Met or Val
  VARIANT 243: note = Xaa is Gly or Glu
  VARIANT 337: note = Xaa is Pro or Leu
  VARIANT 353: note = Xaa is Ile or Val
  VARIANT 424: note = Xaa is Gln or Leu
  VARIANT 428: note = Xaa s Lys or Asn
  VARIANT 432: note = Xaa is Asn or Asp
  VARIANT 436: note = Xaa is Ser or Gly
  VARIANT 465: note = Xaa is Asn or Lys
  VARIANT 492: note = Xaa is Lys or Ile
  VARIANT 498: note = Xaa is Asn or Ser
  VARIANT 500: note = Xaa is Ser or Leu
  VARIANT 502: note = Xaa is Lys or Arg
  VARIANT 518: note = Xaa is Gln, Pro, or Arg
  VARIANT 521: note = Xaa is Phe or Leu
MSSLASNRXLTLPQRTXRGKNKHCWSTSKXTRRSRVSALXIVRSQRGPTRMCRNIYDPLLCFKLFFTDEIISEIVKWTNAEISLKRRESMTXATFRDTNEDEIYAFFGILVXTAVRKDNHXSTDDLFDRSLSMVYVSVMSRDRFDFLIRCLRMDDKXIRPTLRENDVFTPVRKIWDLFIHQCIQNYTPGAHLTIDEQLLGFRGRCPFXXYIPNKPSKYGIKILMMCDSGTKYMINGMPYLGRXTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKXAKMVYLLSSCDEDASXNESTGKPQMVMYYNQTKGGVDTLDQMCSVMTCSRKTNRWPMALLYGMINIACINSFIIYSHNVSSKGEKVXSRKXFMRXLYMXLTSSFMRKRLEAPTLKRYLRDNISNILPXEVPGTSDDSTEEPVMKKRTYCTYCPSXIRRKAXAXCXKCKKVICREHNIDMCXSCX

SEQ ID NO: 12 - protein, 646 aa, synthetic construct
  VARIANT 1: note = Xaa is Gly or Cys
  VARIANT 3: note = Xaa is Ser or Asn
  VARIANT 25: note = Xaa is Ser or Pro
  VARIANT 29: note = Xaa is Ile or Val
  VARIANT 39: note = Xaa is Gln or Arg
  VARIANT 42: note = Xaa is Thr or Ala
  VARIANT 54: note = Xaa is Gln or Arg
  VARIANT 56: note = Xaa is Thr or Ala
  VARIANT 60: note = Xaa is Ser or Arg
  VARIANT 81: note = Xaa is Ile or Val
  VARIANT 89: note = Xaa is Ile or Val
  VARIANT 102: note = Xaa is Ser, Pro, or Thr
  VARIANT 112: note = Xaa is Asn or Ser
  VARIANT 164: note = Xaa is Gly or Ser
  VARIANT 184: note = Xaa is Met or Leu
  VARIANT 193: note = Xaa is Met or Val
  VARIANT 229: note = Xaa is Ser or Asn
  VARIANT 280: note = Xaa is Arg or Gly
  VARIANT 281: note = Xaa is Met or Val
  VARIANT 315: note = Xaa is Gly or Gln
  VARIANT 409: note = Xaa is Pro or Leu
  VARIANT 425: note = Xaa is Ile orVal
  VARIANT 496: note = Xaa is Gln or Leu
  VARIANT 500: note = Xaa is Lys or Asn
  VARIANT 504: note = Xaa is Asn or Asp
  VARIANT 508: note = Xaa is Ser or Gly
  VARIANT 537: note = Xaa is Asn or Lys
  VARIANT 564: note = Xaa is Lys or Ile
  VARIANT 570: note = Xaa is Asn or Ser
  VARIANT 572: note = Xaa is Ser or Leu
  VARIANT 574: note = Xaa is Lys or Arg
  VARIANT 590: note = Xaa is Gln, Pro, or Arg
  VARIANT 593: note = Xaa is Phe or Leu
  VARIANT 617: note = Xaa is Lys or Ile
  VARIANT 623: note = Xaa is Asn or Ser
  VARIANT 625: note = Xaa is Ser or Leu
  VARIANT 627: note = Xaa is Lys or Arg
  VARIANT 643: note = Xaa is Gln, Pro, or Arg
  VARIANT 646: note = Xaa is Phe or Leu
XSXLDDEHILSALLQSDDELVGEDXDSEXSDHVSEDDVXSDXEEAFIDEVHEVXPXSSGXEILDEQNVIEQPGSSLASNRXLTLPQRTXRGKNKHCWSTSKXTRRSRVSALXIVRSQRGPTRMCRNIYDPLLCFKLFFTDEIISEIVKWTNAEISLKRRESMTXATFRDTNEDEIYAFFGILVXTAVRKDNHXSTDDLFDRSLSMVYVSVMSRDRFDFLIRCLRMDDKXIRPTLRENDVFTPVRKIWDLFIHQCIQNYTPGAHLTIDEQLLGFRGRCPFXXYIPNKPSKYGIKILMMCDSGTKYMINGMPYLGRXTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKXAKMVYLLSSCDEDASXNESTGKPQMVMYYNQTKGGVDTLDQMCSVMTCSRKTNRWPMALLYGMINIACINSFIIYSHNVSSKGEKVXSRKXFMRXLYMXLTSSFMRKRLEAPTLKRYLRDNISNILPXEVPGTSDDSTEEPVMKKRTYCTYCPSXIRRKAXAXCXKCKKVICREHNIDMCXSCXGTSDDSTEEPVMKKRTYCTYCPSXIRRKAXAXCXKCKKVICREHNIDMCXSCX

SEQ ID NO: 13 - protein, 574 aa, synthetic construct
  VARIANT 9: note = Xaa is Ile or Val
  VARIANT 17: note = Xaa is Ile or Val
  VARIANT 30: note = Xaa is Ser, Pro, or Thr
  VARIANT 40: note = Xaa is Asn or Ser
  VARIANT 92: note = Xaa is Gly or Ser
  VARIANT 112: note = Xaa is Met or Leu
  VARIANT 121: note = Xaa is Met or Val
  VARIANT 157: note = Xaa is Ser or Asn
  VARIANT 208: note = Xaa is Arg or Gly
  VARIANT 209: note = Xaa is Met or Val
  VARIANT 243: note = Xaa is Gly or Glu
  VARIANT 337: note = Xaa is Pro or Leu
  VARIANT 353: note = Xaa is Ile or Val
  VARIANT 424: note = Xaa is Glu or Leu
  VARIANT 428: note = Xaa is Lys or Asn
  VARIANT 432: note = Xaa is Asn or Asp
  VARIANT 436: note = Xaa is Ser or Gly
  VARIANT 465: note = Xaa is Asn or Lys
  VARIANT 492: note = Xaa is Lys or Ile
  VARIANT 498: note = Xaa is Asn or Ser
  VARIANT 500: note = Xaa is Ser or Leu
  VARIANT 502: note = Xaa is Lys or Arg
  VARIANT 518: note = Xaa is Glu, Pro, or Arg
  VARIANT 521: note = Xaa is Phe or Leu
  VARIANT 545: note = Xaa is Lys or Ile
  VARIANT 551: note = Xaa is Asn or Ser
  VARIANT 553: note = Xaa is Ser or Leu
  VARIANT 555: note = Xaa is Lys or Arg
  VARIANT 571: note = Xaa is Glu, Pro, or Arg
  VARIANT 574: note = Xaa is Phe or Leu
MSSLASNRXLTLPQRTXRGKNKHCWSTSKXTRRSRVSALXIVRSQRGPTRMCRNIYDPLLCFKLFFTDEIISEIVKWTNAEISLKRRESMTXATFRDTNEDEIYAFFGILVXTAVRKDNHXSTDDLFDRSLSMVYVSVMSRDRFDFLIRCLRMDDKXIRPTLRENDVFTPVRKIWDLFIHQCIQNYTPGAHLTIDEQLLGFRGRCPFXXYIPNKPSKYGIKILMMCDSGTKYMINGMPYLGRXTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKXAKMVYLLSSCDEDASXNESTGKPQMVMYYNQTKGGVDTLDQMCSVMTCSRKTNRWPMALLYGMINIACINSFIIYSHNVSSKGEKVXSRKXFMRXLYMXLTSSFMRKRLEAPTLKRYLRDNISNILPXEVPGTSDDSTEEPVMKKRTYCTYCPSXIRRKAXAXCXKCKKVICREHNIDMCXSCXGTSDDSTEEPVMKKRTYCTYCPSXIRRKAXAXCXKCKKVICREHNIDMCXSCX

SEQ ID NO: 14 - protein, 490 aa, synthetic construct
RRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 15 - protein, 104 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKST

SEQ ID NO: 16 - protein, 490 aa, synthetic construct; identical to SEQ ID NO: 14

SEQ ID NO: 17 - protein, 490 aa, synthetic construct; identical to SEQ ID NO: 14

SEQ ID NO: 18 - protein, 543 aa, synthetic construct
RRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 19 - protein, 13 aa, synthetic construct
MAYPYDVPDYATS

SEQ ID NO: 20 - protein, 490 aa, synthetic construct; identical to SEQ ID NO: 14

SEQ ID NO: 21 - protein, 503 aa, synthetic construct
MAYPYDVPDYATSRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 22 - protein, 490 aa, synthetic construct; identical to SEQ ID NO: 14

SEQ ID NO: 23 - protein, 556 aa, synthetic construct
MAYPYDVPDYATSRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFGTSDDSTEEPVMKKRTYCTYCPSKIRRKANASCKKCKKVICREHNIDMCQSCF

SEQ ID NO: 24 - protein, 567 aa, synthetic construct
MSQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKN

SEQ ID NO: 25 - protein, 527 aa, synthetic construct
VMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKN

SEQ ID NO: 26 - protein, 647 aa, synthetic construct
MSQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNSTSGMRAKPPTSDPPCRLSMDMRKHTLQAIVGSGKKKNILRRCRVCSVHKLRSETRYMCKFCNIPLHKGACFEKYHTLKN

SEQ ID NO: 27 - protein, 607 aa, synthetic construct
VMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNSTSGMRAKPPTSDPPCRLSMDMRKHTLQAIVGSGKKKNILRRCRVCSVHKLRSETRYMCKFCNIPLHKGACFEKYHTLKN

SEQ ID NO: 28 - DNA, 35 bp, synthetic construct
  source 1..35: mol_type = other DNA
ccctagaaagatagtctgcgtaaaattgacgcatg

SEQ ID NO: 29 - DNA, 63 bp, synthetic construct
  source 1..63: mol_type = other DNA
ccctagaaagataatcatattgtgacgtacgttaaagataatcatgcgtaaaattgacgcatg

SEQ ID NO: 30 - protein, 45 aa, synthetic construct
MGCSLDDEHILSALLQSDDELVGEDSDSEISDHVSEDDVQSDTEE

SEQ ID NO: 31 - protein, 41 aa, synthetic construct
MSQHSDYSDDEFCADKLSNYSCDSDLENASTSDEDSVMVRP

SEQ ID NO: 32 - DNA, 10 bp, synthetic construct
  source 1..10: mol_type = other DNA
ctagggttaa

SEQ ID NO: 33 - protein, 647 aa, unidentified
MSQHSDYADDEFCADKLSNYSCDADLENASTADEDSADDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTWSPYTDSWYHIYMDNYYNSVANCEALMKNKFRICGTIRKNRGIPKDFQTISLKKGETKFIRKNDILLQVWQSKKPVYLISSHSAEMEESQNIDRTSKKKIVKPNALIDYNKHMKGVDRADQYLSYYSILRRWKWTKRLAMYMINCALFNSYAVYKSVRQRKMGFKMFLKQTAHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNSTSGMRAKPPTSDPPCRLSMDMRKHTLQAIVGSGKKKNILRRCRVCSVHKLRSETRYMCKFCNIPLHKGACFEKYHTLKN

SEQ ID NO: 34 - DNA, 153 bp, unidentified
  source 1..153: mol_type = other DNA
cacttggattgcgggaaacgagttaagtcggctcgcgtgaattgcgcgtactccgcgggagccgtcttaactcggttcatatagatttgcgtggagtgcgggaaacgtgtaaactcgggccgattgtaactgcgtattaccaaattatttgtt

SEQ ID NO: 35 - DNA, 208 bp, unidentified
  source 1..208: mol_type = other DNA
cactacggtgtcgggtgaatttcccgccaaaatataagatggcgcgggcaattatttaggtttcccgcgcaaaatcagagttagtttaaaatgcctatatcttttgaagtatgggttcgattgaaatgaaattttcggtttcttgtgtaaaaaatgaaaatttattcaatcacagacatttttttatctattcagtacataaataatt

SEQ ID NO: 36 - protein, 45 aa, unidentified
MSQHSDYSDDEFCADKLSNYSCDSDLENASTSDEDSSDEDVMVRP

SEQ ID NO: 37 - protein, 45 aa, unidentified; identical to SEQ ID NO: 30